(19) United States
(12) Patent Application Publication
(10) Pub. No.: US 2019/0142835 A1
(43) Pub. Date: May 16, 2019

(54) INHIBITION OF AUTOPHAGY USING PHOSPHOLIPASE A2 INHIBITORS

(71) Applicant: OREGON HEALTH & SCIENCE UNIVERSITY, PORTLAND, OR (US)

(72) Inventors: George Thomas, Portland, OR (US); Jennifer Podolak, Portland, OR (US); Hui-wen Lue, Portland, OR (US); Kevin Kolahi, Portland, OR (US)

(73) Assignee: OREGON HEALTH & SCIENCE UNIVERSITY, PORTLAND, OR (US)

(21) Appl. No.: 16/191,106

(22) Filed: Nov. 14, 2018

Related U.S. Application Data
(60) Provisional application No. 62/585,546, filed on Nov. 14, 2017.
(60) Provisional application No. 62/586,066, filed on Nov. 14, 2017.

(51) Int. Cl. A61K 31/519; A61K 31/5377; A61K 31/4709; A61P 35/00

(52) U.S. Cl. CPC A61K 31/519; A61P 35/00; A61K 31/4709; A61K 31/5377

(57) ABSTRACT

Provided are methods and pharmaceutical combinations utilizing a phospholipase A2 inhibitor for the inhibition of treatment-induced autophagy.

### RELATED APPLICATIONS

This application claims priority to U.S. Provisional Appln. Nos. 62/585,546 and 62/586,066, both filed Nov. 14, 2017.

### ACKNOWLEDGEMENT OF GOVERNMENT SUPPORT

This invention was made with government support under CA169172 awarded by the National Institutes of Health. The government has certain rights in the invention.

### FIELD OF THE INVENTION

Provided are methods and pharmaceutical combinations to enhance oncology outcomes in pharmaceutical treatments that induce autophagy. More specifically, methods and pharmaceutical combinations utilizing a phospholipase A2 inhibitor to inhibit treatment-induced autophagy.

### BACKGROUND OF THE INVENTION

Cancer cells that survive despite initial response to drugs that are tailored to specifically inhibit oncogenic signaling pathways is a constant in clinical oncology, with lethal consequences. This reservoir of cancer cells that survive the first-line treatment are clinically termed residual disease and serve as the nidus for the eventual emergence of acquired resistance [1,2]. Response rates to second- and third-line therapies in the setting of resistance are progressively lower because these patients now have poorer performance status, additional co-morbidities, and are less able to tolerate side effects.

Therefore, the best chance of achieving cures or long-term control of metastatic disease is at the time of first-line therapies. However, the current knowledge of the underlying tumor cell adaptations and how they survive during this initial treatment is limited. Oncogenic growth factor signaling can be distilled to a singular, unifying purpose: to marshal the nutrient uptake required to meet the cancer cell's unrelenting metabolic demands for growth. Therapies that inhibit growth factor signaling negatively impact the nutrient supply chain and consequently, tumor cell survival and fitness. Accordingly, deciphering how cancer cells survive despite treatment-induced nutrient depletion can potentially inform novel therapeutic approaches capable of killing all cancer cells at the time of initial treatment, and subsequently translate into durable clinical responses. One survival mechanism that both normal and cancer cells utilize is autophagy. Autophagy is an evolutionary conserved catabolic process by which cells survive nutrient deprivation by sequestering regions of the cytosol and organelles in double membrane vesicles known as autophagosomes, which then fuse with lysosomes and are degraded [3,4]. Paradoxically, cancer therapies increase autophagic rates, though the molecular basis for this is not well understood. While residual disease in solid tumors is very likely due to a mixed set of mechanisms, we reasoned that at its root are cancer cells that can rewire their signaling and metabolic networks to adapt to treatment-imposed metabolic restrictions. We hypothesized that these cancer cells relied on autophagy to survive. This hypothesis was based on the knowledge that nutrients derived from autophagic degradation are reutilized to maintain macromolecular synthesis and or oxidized to maintain bioenergetics [5].

There remains a need for treatments that will inhibit autophagy activity and cancer cell survival.

### SUMMARY OF THE INVENTION

Tumor cells that survive first-line cancer therapies are clinically defined as “residual disease” and are the primary cause of relapse in all cancers. Yet how these persistent cancer cells survive is largely unknown. Autophagy occurs at a basal rate in most cells to maintain cellular metabolic homeostasis, and cancer cells increase autophagy to supply nutrients required for their survival and growth.

Paradoxically, cancer therapies also increase autophagy, though the mechanisms responsible for this are unclear. Herein are provided first-line treatments that effectively shuts down PI3K-AKT-mTOR signaling, markedly decrease glycolysis, and restrain tumor growth. However, these metabolic restrictions triggered autophagic catabolism of phospholipids which supplied the metabolites required for the maintenance of mitochondrial respiration and redox homeostasis, thereby enabling cancer cell survival. Specifically, survival of treated cancer cells was critically dependent on phospholipase A2 (PLA2) to mobilize lysophospholipids and free fatty acids to support fatty acid oxidation and oxidative phosphorylation.

Accordingly, pharmacologic inhibition of PLA2 decreased oxidative phosphorylation and correspondingly, increased apoptosis. Together, these studies establish that therapy-enforced metabolic restrictions, while restraining tumor growth, reciprocally activates autophagy as a salvage pathway to support residual disease. Importantly, we identify PLA2 as tractable metabolic target to eradicate residual disease.

Provided herein is a method of inhibiting autophagy in a human receiving a pharmaceutical agent that induces autophagy, the method comprising administering to a human in need thereof a pharmaceutically effective amount of a phospholipase A2 inhibitor, or a pharmaceutically acceptable salt thereof.

Also provided is a method of treatment for treatment-induced autophagy in a human, the method comprising administering to a human in need thereof a pharmaceutically effective amount of a phospholipase A2 inhibitor.

Further provided is a method of inhibiting cancer cell survival in a human experiencing treatment-induced autophagy, the method comprising administering to a human in need thereof a pharmaceutically effective amount of a phospholipase A2 inhibitor, or a pharmaceutically acceptable salt thereof.

### BRIEF DESCRIPTION OF THE SEVERAL VIEWS OF THE DRAWINGS

This application contains at least one drawing executed in color. Copies of this application with color drawing(s) will be provided by the Office upon request and payment of the necessary fee. At least some of the drawings submitted herein are better understood in color. Applicant considers the color versions of the drawings as part of the original submission and reserve the right to present color images of the drawings in later proceedings. Applicant hereby incorporates by reference the color drawings filed herewith and retained in SCORE. The attached drawings are for purposes of illustration and are not necessarily to scale.

FIG. 1A is a set of bar graphs showing cell viability when CYT387 is used in combination with GDC0941, BX795, and MK2206 in a human ACHN cell line.

FIG. 1B is a set of bar graphs showing an indicator of apoptosis as measured by cleaved-caspase 3/7 changes when CYT387 is used in combination with GDC0941, BX795, and MK2206 in a human ACHN cell line.

FIG. 1C is a set of bar graphs showing cell viability when CYT387 is used in combination with GDC0941, BX795, and MK2206 in a human SN12C cell line.

FIG. 1D is a set of bar graphs showing an indicator of apoptosis as measured by cleaved-caspase 3/7 changes when CYT387 is used in combination with GDC0941, BX795, and MK2206 in a human SN12C cell line.

FIG. 2A is a plot of tumor volume over time for ACHN xenografts treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p<0.01****)

FIG. 2B is a boxplot of apoptosis response in ACHN xenograft tumors treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p<0.0001).

FIG. 2C is a boxplot of proliferation response in ACHN xenograft tumors treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p=0.0018).

FIG. 2D is a plot of tumor volume over time for SN12C xenografts treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p<0.0001****)

FIG. 2E is a boxplot of apoptosis response in SN12C xenograft tumors treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p<0.0001).

FIG. 2F is a boxplot of proliferation response in SN12C xenograft tumors treated with Vehicle, CYT387 (50 mg/kg), MK2206 (60 mg/kg) and CYT387-MK2206 (50 mg/kg+60 mg/kg) combination. Error bars represent mean±SEM. (Control vs CYT387+MK2206 p<0.0001)).

FIG. 2G is a plot showing stable mouse weights with treatment: Vehicle, CYT387 (50 mg/kg), MK2206 (60 mg/kg) and CYT387-MK2206 (50 mg/kg+60 mg/kg) combination. Body weights of mice bearing ACHN tumors as indicated. Data are presented as mean±SEM; ns: not significant.

FIG. 2H is a plot showing stable mouse weights with treatment: Vehicle, CYT387 (50 mg/kg), MK2206 (60 mg/kg) and CYT387-MK2206 (50 mg/kg+60 mg/kg) combination. Body weights of mice bearing SN12C tumors as indicated. Data are presented as mean±SEM; ns: not significant.

FIG. 2I is a set of photomicrographs of tumor tissue from ACHN xenografts treated with the indicated drug regimens and evaluated by immunofluorescence for p-S6 and p-AKT

FIG. 3A is a bar graph showing the treatment effect of control, CYT387, MK2206, CYT387+MK2206 on glucose uptake over time, measured by 18FDG.

FIG. 3B is a set of bar graphs showing glucose and lactate levels in culture media as measured in control and treated cells, normalized to cell number.

FIG. 3C is a bar graph showing cell diameter changes of ACHN cells treated with CYT387, MK2206, CYT387+MK2206 or vehicle (DMSO). (* p<0.02)

FIG. 3D is a plot ECAR (an indicator of glycolysis) over time in ACHN cells as measured using a XF-96 Extracellular Flux Analyzer after pre-incubation with drugs or DMSO. Shown are ECAR means±SD of experimental triplicates.

FIG. 3E is a bar graph showing the effect of treatment on basal ECAR, measured in real time and presented as change in mpH per unit time (representative results shown, n=2).

FIG. 3F is a bar graph showing OCR/ECAR ratios of treated ACHN cells (representative results shown, n=2).

FIG. 3G is a bar graph of data showing that treatment activates p-AMPK and increases NADPH levels. ACHN cells were treated with control, 2 μM CYT387, 10 μM MK2206, CYT387+MK2206 for 24 hr.

FIG. 3H is a bar graph of data showing that treatment maintains GSSG/GSH ratios. ACHN cells were treated with control, 2 μM CYT387, 10 μM MK2206, CYT387+MK2206 for 24 hr.

FIG. 3I is a bar graph of data showing that treatment mitigates ROS. ACHN cells were treated with control, 2 μM CYT387, 10 μM MK2206, CYT387+MK2206 for 24 hr.

FIG. 4A shows a set of representative images from an experiment where ACHN cells were treated with control, CYT387, MK2206, CYT387+MK2206 for 24 hrs, then Bodipy 493/503 (green) added to visualize lipid droplets (n=5 experiments). These experiments show that autophagy drives lipid droplet growth during nutrient depletion.

FIG. 4B is a bar graph of data showing the increase in the number of lipid droplets for the indicted treatments. Data are expressed as means±SEM. *p<0.001 for Control vs CYT387, control vs MK2206, control vs CYT387+MK2206.

FIG. 4C is a bar graph of data showing the increase in the size of lipid droplets for the indicted treatments. Data are expressed as means±SEM. *p<0.001 for Control vs CYT387, control vs MK2206, control vs CYT387+MK2206.

FIG. 4D is a bar graph of data showing the increase in lipid drops in vivo using adipophilin staining in xenograft tumors (n=9). Data are expressed as means±SEM. *p<0.001, control vs CYT387, control vs MK2206, control vs CYT387+MK2206. Measured in tumors resected after 40 days of treatment.

FIG. 4E is a bar graph of data showing the mean IF intensity for the indicated treatments. Atg5+/+ murine embryonic fibroblasts were treated with 2 μM CYT387, 10M MK2206 and the combination for 24 hr. Bodipy was added and the lipid droplet number was measured. n=500 cells, *p<0.001 control vs CYT387, control vs CYT387+MK2206, p<0.005 for control vs MK2206.

FIG. 4F is a bar graph of data showing the mean IF intensity for the indicated treatments. Atg5−/− murine embryonic fibroblasts were treated with 2 μM CYT387, 10 μM MK2206 and the combination for 24 hr. Bodipy was added and the lipid droplet number was measured. n=500 cells, p=NS: no significance between treatment groups.

FIG. 4G is a set of representative immunofluorescence images and a bar graph of mitochondria quantification (n=5 experiments). ACHN cells were treated with control, CYT387, MK2206, CYT387+MK2206 for 24 hrs, and Mitotracker Orange was added to visualize mitochondria. Mitochondria number was measured, and data is expressed as means±SEM. *p<0.001 control v CYT, control v MK, control vs MK+CYT.

FIG. 4H shows a representative immunofluorescence image highlighting the spatial distribution of lipid droplets and mitochondria. The dual staining of Bodipy and Mitotracker Orange demonstrate close proximity of lipid droplets with mitochondria in CYT387+MK2206 co-treated ACHN cells.

FIG. 4I shows a graphical representation of a metabolite profiling to assess the effect of treatment on lipids (decrease, increase, or no change). Global metabolite profiling reveals a preferential decrease in lipids. Decrease: abundance less than 0.5-fold in treated cells compared to the vehicle. Increase: abundance greater than 2-fold in treated cells compared to the vehicle. In the lower portion of the figure, a bar graph of data is shown for the measurement of lipid driven OCR, measured by acute inhibition of CPT-1 with etomoxir (*p<0.01).

FIG. 5A is a set of representative immunofluorescence images of ACHN cells treated with control, OOEPC, CYT387, CYT387+OOEPC, MK2206, MK2206+OOEPC, CYT387+MK2206, CYT387+MK2206+OOEPC for 24 hrs. Bodipy 493/503 (green) was added to visualize lipid droplets (n=3 experiments).

FIG. 58B is a bar graph of data showing the number of lipid droplets for the indicated treatments. Data are expressed as means±SEM. *p<0.0001 CYT387 v CYT387+OOEPC, MK2206 v MK2206+OOEPC, CYT387+MK2206 v CYT387+MK2206+OOEPC.

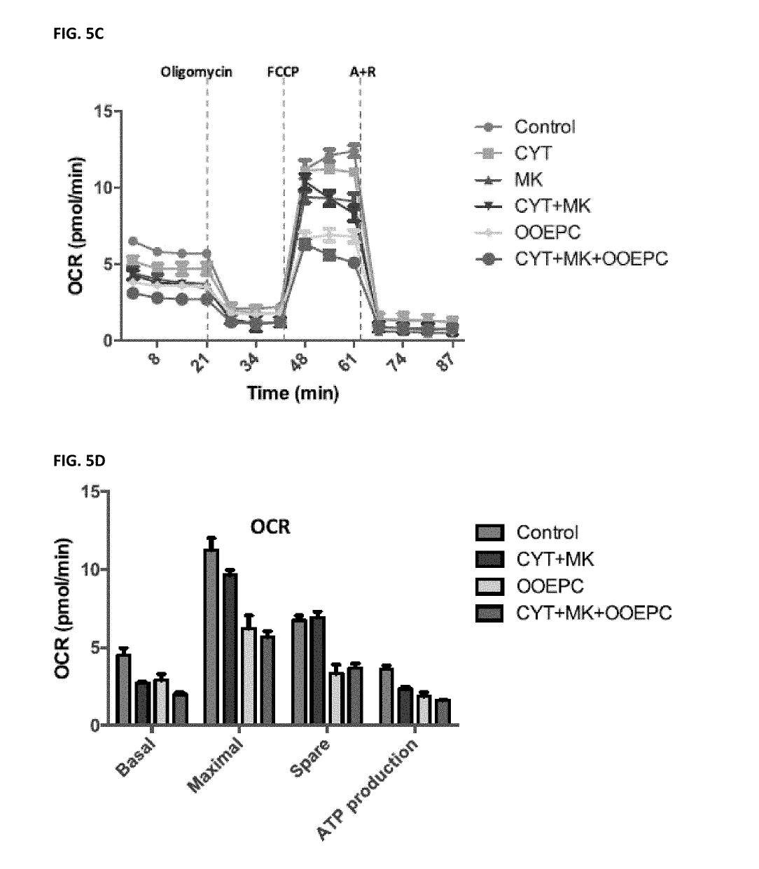

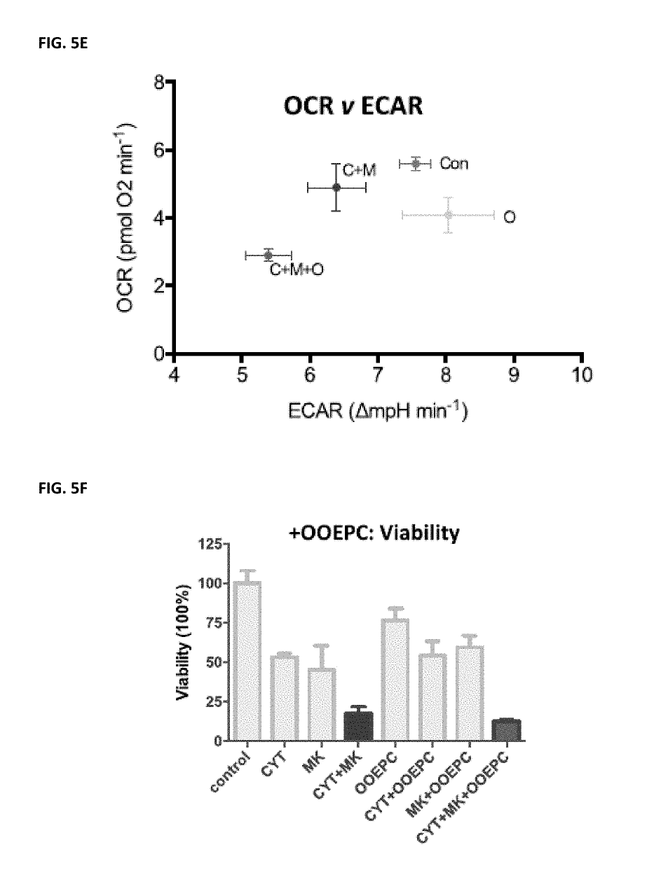

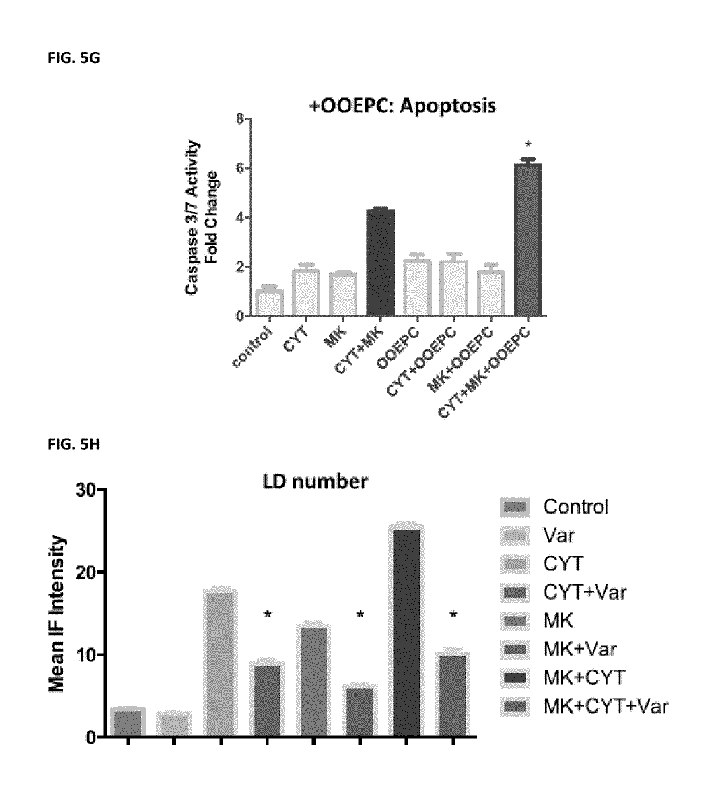

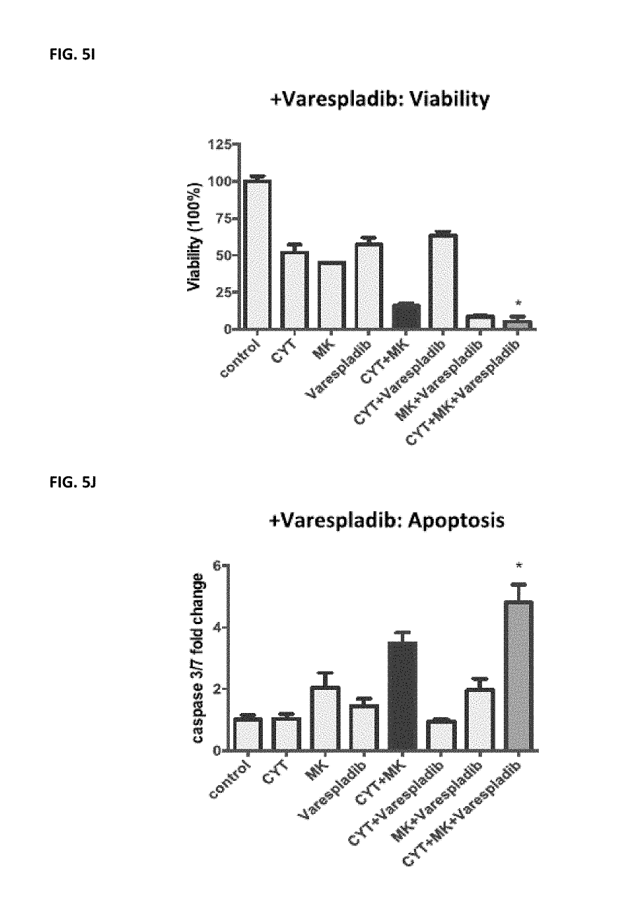

FIG. 5C is a plot of oxygen consumption rate (OCR) over time for ACHN cells that were treated with DMSO (control), OOEPC, CYT387, CYT387+OOEPC, MK2206, MK2206+OOEPC, CYT387+MK2206, CYT387+MK2206+OOEPC for 24 h. OCR was determined using a XF-96 Extracellular Flux Analyzer during sequential treatments with oligomycin, FCCP, and rotenone/antimycin (A+R).

FIG. 5D is a set of bar plots showing initial basal OCR, maximal OCR, Spare respiratory capacity (SRC: the quantitative difference between maximal uncontrolled OCR and the initial basal OCR), and ATP production. Shown are OCR means±SD of experimental triplicates. For ease of viewing, only control, OOEPC, CYT387+MK2206, CYT387+MK2206+OOEPC data is graphed.

FIG. 5E is a plot showing OCR versus ECAR (means±SEM, experimental triplicates) after the addition of OOEPC to the CYT387-MK2206 combination (Con: Control; O: OOEPC; C+M: CYT387+MK2206; C+M+O: CYT387+MK2206+OOEPC).

FIG. 5F is a bar plot showing cell viability data when OOEPC is added to each of the treatment groups CYT387, MK2206, CYT387+MK2206 (n=3). Data are expressed as means±SD (CYT387+MK2206 vs CYT387+MK2206+OOEPC: p=ns).

FIG. 5G is a bar plot showing Caspase3/7 activity data when OOEPC is added to each of the treatment groups CYT387, MK2206, CYT387+MK2206 (n=3). Data are expressed as means±SD (CYT387+MK2206 vs CYT387+MK2206+OOEPC: p<0.001, ***).

FIG. 5H is a bar plot showing the effect of adding Varespladib, a distinct PLA2 inhibitor, to CYT387, MK2206, CYT387+MK2206 on lipid droplet numbers. Bodipy staining was used.

FIG. 5I is a bar plot showing cell viability data when Varespladib is added to each of the treatment groups CYT387, MK2206, CYT387+MK2206 (n=3). Data are expressed as means±SD. (CYT387+MK2206 vs CYT387+MK2206+Varespladib: p<0.01, **).

FIG. 5J is a bar plot showing Caspase3/7 activity data when Varespladib is added to each of the treatment groups CYT387, MK2206, CYT387+MK2206 (n=3). Data are expressed as means±SD. (CYT387+MK2206 vs CYT387+MK2206+Varespladib: p<0.1, *).

### DETAILED DESCRIPTION OF THE INVENTION

Also provided is a method of inhibiting treatment-induced autophagy in a human, the treatment-induced autophagy resulting from the human receiving a pharmaceutical agent selected from the group of a Janus Kinase (JAK) inhibitor, VEGF/VEGFR receptor tyrosine kinase inhibitor, a protein kinase A (PKA) inhibitor, a multi-kinase inhibitor, a phosphoinositide 3-kinase (PI3K) inhibitor, an AKT inhibitor (such as MM-2206), a mechanistic target of rapamycin (mTOR) inhibitor, a protein kinase C (PKC) inhibitor, a mitogen-activated protein kinase kinase (MEK) inhibitor, a CDK9 inhibitor, and a proteasome inhibitor, the method comprising administering to a human in need thereof a pharmaceutically effective amount of a phospholipase A2 inhibitor, or a pharmaceutically acceptable salt thereof.

Phospholipase A2 (PLA2) inhibitors useful in the methods herein include anagrelide (AGRLIN) and cilostazol (PLETAL). Anagrelide may be administered at a dose of from about 0.5 mg to about 20 mg per dose. Cilostazol may be administered at a dose of from about 50 mg to about 250 mg per dose. Other PLA2 inhibitors for use in the methods herein include varespladib, Darapladib, ulobetasol, oleyloxyethyl phosphorylcholine, cytidine 5-prime-diphosphocholine sodium salt (CDP-choline), U-73122, quinacrine dihydrochloride, quercetin dihydrate, chlorpromazine HCl, aristolochic acid, cynnamycin, MJ33, ETYA, N-(p-amylcinnamoyl)anthranilic acid (ACA), isotetrandrine, quinacrine dihydrochloride dihydrate, YM 26734, dihydro-D-erythro-sphingosine, PACOCF3, ONO-RRS-082, Luffariellolide, RSC-3388, LY 311727, OBAA, AX 048, 2-Hydroxy-1,1,1,-trifluoro-6,9,12,15-heneicosatetraene (AACH(OH)CF3), 2-oxo-1,1,1-Trifluoro-6,9-12,15-heneicosatetraene (AACOCF3), 2-oxo-6,9,12,15-Heneicosatetetraene (AACOCH3), (E)-6-(Bromomethylene)tetrahydro-3-(1-naphthalenyl)-2H-pyran-2-one (BEL, B1552), 4,7,10,13-Nonadecatetraenyl fluorophosphonic acid methyl ester (MAFP), N-[6-(4-Chlorophenyl)hexyl]-2-oxo-4-[(S)-(phenylmethyl)sulfinyl]-1-azetidineacetamide (SB-222657), N-[6-(4-Chlorophenyl)hexyl]-2-oxo-4-[(R)-(phenylmethyl)sulfinyl]-1-azetidineacetamide (SB-223777), Palmityl trifluoromethylketone (PACOCF3, CAS 141022-99-3), and (S)-bromoenol lactone ((S)-BEL).

Additional PLA2 inhibitors that may be used in the present application include darapladib (SB-480848, CAS#356057-34-6), N-(2-diethylamino-ethyl)-2-[2-(4-fluoro-benzylsulfanyl)-4-oxo-4,5,6,7-tetra hydro-cyclopenta pyrimidin-1-yl]-N-(4′-trifluoromethyl-biphenyl-4-ylmethyl)-acetamide; SB435495; GSK-2647544; varespladib; mepacrine bromophenylbromide; and the pyrimidinone inhibitors of lipoprotein-associated PLA2 taught in U.S. Pat. No. 9,585,884, the full contents of which are incorporated here in reference.

It will be understood that the oncology agents listed herein may be used in the methods herein in the doses and regimens for which they are known for each of the cancers, tumors, and malignancies referred to in the methods.

VEGF/VEGFR inhibitors useful in the methods herein include pazopanib (VOTRIENT®), bevacizumab (AVASTIN®), sunitinib (SUTENT®), sorafenib (NEXAVAR®), axitinib)(INLYTA®), regorafenib (STIVARGA®), ponatinib (ICLUSIG®), cabozantinib, vandetanib, ramucirumab, lenvatinib, and ziv-aflibercept.

Pazopanib may be administered in the methods herein at a daily dose of from about 0.1 mg/kg to about 10 mg/kg. In some embodiments, pazopanib may be administered at a daily dose of from about 1 mg/kg to about 5 mg/kg. In other embodiments, pazopanib may be administered at a daily dose of from about 1 mg/kg to about 3 mg/kg.

Bevacizumab may be administered in the methods herein at a dose of from about 1 mg to about 20 mg. In some embodiments, the dose of bevacizumab may be from about 2.5 mg to about 150 mg. In separate embodiments, bevacizumab may be administered at about 5 mg, about 7.5 mg, about 10 mg, about 12.5 mg, and about 15 mg. In some embodiments, the doses listed for bevacizumab herein are administered to a subject in need thereof once every two weeks. In other embodiments, the doses are administered once every three weeks.

Sunitinib may be administered orally in the methods herein at a daily dose of from about 5 mg to about 75 mg. In some embodiments, the dose of sunitinib may be from about 12.5 mg to about 50 mg. In some embodiments, the daily dose of sunitinib is from about 20 mg to about 30 mg. In some separate embodiments, the daily dose of sunitinib is 12.5 mg/day, 25 mg/day, and 50 mg, respectively.

Sorafenib may be administered orally in the methods herein at a daily dose of from about 100 mg to about 400 mg taken once or twice daily. In some embodiments, the dose of sunitinib may be from about 100 mg to about 300 mg taken once or twice daily. In other embodiments, the dose of sunitinib may be from about 100 mg to about 200 mg taken once or twice daily.

Axitinib may be administered orally in the methods herein at a dose of from about 1 mg to about 10 mg taken once or twice daily. In other embodiments, axitinib is administered orally at a daily dose of from about 1 mg to about 7 mg taken once or twice daily. In other embodiments, axitinib is administered orally at a daily dose of from about 1 mg to about 5 mg taken once or twice daily.

Regorafenib may be administered orally in the methods herein at a dose of from about 40 mg to about 200 mg taken once or twice daily. In separate embodiments, regorafenib may be administered to a subject in need thereof at daily doses of 40 mg, 80 mg, 120 mg, 160 mg, and 200 mg, respectively.

VEGF/VEGFR inhibitor ponatinib may be administered in the methods herein orally at doses of from about 10 mg to about 100 mg daily. In some embodiments, ponatinib is administered at a dose range of from about 15 mg to about 60 mg daily. In some embodiments, ponatinib is administered in a dose of 45 mg.

Cabozantinib may be administered in the methods herein orally at doses of from about 10 mg to about 100 mg daily. In some embodiments, cabozantinib is administered at a dose range of from about 20 mg to about 80 mg daily. In other embodiments, cabozantinib is administered at a dose range of from about 20 mg to about 60 mg daily.

Vandetanib may be administered in the methods herein orally at doses of from about 100 mg to about 500 mg daily. In some embodiments, vandetanib is administered at a dose range of from about 100 mg to about 400 mg daily. In other embodiments, vandetanib is administered at a dose range of from about 200 mg to about 300 mg daily.

Ramucirumab may be administered by infusion in the methods herein at from about 5 mg/kg to about 10 mg/kg once every two weeks. In some embodiments, ramucirumab may be administered at a dose of about 8 mg/kg once every two weeks.

Lenvatinib may be administered orally in the methods herein at an oral daily dose of from about 1 mg to about 50 mg. In some embodiments, lenvatinib is administered at a daily dose of from about 4 mg to about 30 mg. In some embodiments, lenvatinib is dosed at about 24 mg/day.

Ziv-aflibercept may be administered by infusion in the methods herein at a dose of from about 1 mg/kg to about 10 mg/kg once every two weeks or once every three weeks. In some embodiments, ziv-aflibercept is administered at a dose of from about 1 mg/kg to about 5 mg/kg once every two weeks. In another embodiment, ziv-flibercept is administered at a dose of from about 5 mg/kg to about 10 mg/kg once every three weeks.

Provided is a method of inhibiting VEGF/VEGFR inhibitor-induced autophagy in a human, the method comprising ad ministering to a human in need thereof a pharmaceutically effective amount of a phospholipase A2 inhibitor, or a pharmaceutically acceptable salt thereof.

Also provided is a method of treating soft tissue sarcoma in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating platinum-resistant recurrent epithelial ovarian, fallopian tube, or primary peritoneal cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating persistent, recurrent, or metastatic cervical cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating metastatic colorectal cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating metastatic HER2-negative breast cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating metastatic renal cell carcinoma in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating glioblastoma in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating non-small cell lung cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating pancreatic neuroendocrine tumors in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating gastrointestinal stromal tumors in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Also provided is a method of treating kidney cancer in a human, the method comprising administering to the human in need thereof:

a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

Janus Kinase (JAK) inhibitors that may be used in the methods herein include momelotinib, ruxolitinib (Jakafi®), tofacitinib (CP-690550), azd1480, and fedratinib (SAR302503), as well as pharmaceutically acceptable salts thereof. In some embodiments herein, momelotinib may be administered at a dosage of from about 25 mg per day to about 400 mg per day. In other embodiments, momelotinib may be administered at a dosage of from about 100 mg per day to about 300 mg per day. In further embodiments, momelotinib may be administered at a dosage of from about 150 mg per day to about 250 mg per day. In some embodiments herein, ruxolitinib may be administered at a dosage of from about 5 mg per day to about 50 mg per day. In other embodiments, ruxolitinib may be administered at a dosage of from about 10 mg per day to about 30 mg per day. In further embodiments, ruxolitinib may be administered at a dosage of from about 15 mg per day to about 25 mg per day.

Provided is a method of inhibiting JAK inhibitor-induced autophagy in a human, the method comprising administering to a human in need thereof a pharmaceutically effective amount of a phospholipase A2 inhibitor, or a pharmaceutically acceptable salt thereof.

Also provided is a method of treating pancreatic ductal adenocarcinoma in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating non-small cell lung cancer in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating metastatic non-small cell lung cancer in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating KRAS-mutated non-small cell lung cancer in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating acute myeloid leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating acute leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating acute lymphoblastic leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating chronic myeloid leukemia (also known as chronic myelogenous leukemia and chronic granulocytic leukemia) in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating chronic lymphocytic leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating HER2-positive breast cancer in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating pre-malignant breast disease in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating Lymphoma in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating Hodgkin's Lymphoma in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating Non-Hodgkin's Lymphoma in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating myeloproliferative neoplasia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating primary myelofibrosis (also known as chronic idiopathic myelofibrosis) in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating post-polycythemia vera myelofibrosis in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating essential thrombocythemia myelofibrosis in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating post-essential thrombocythemia myelofibrosis in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating chronic neutrophilic leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treating chronic eosinophilic leukemia in a human, the method comprising administering to the human in need thereof:

a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.
Examples of proteasome inhibitors useful in the methods herein include bortezomib (VELCADE®), marizomib, oprozomib, and delanzomib), as well as pharmaceutically acceptable salts thereof.

Provided is a method of inhibiting proteasome inhibitor-induced autophagy in a human, the method comprising ad ministering to a human in need thereof a pharmaceutically effective amount of a phospholipase A2 inhibitor, or a pharmaceutically acceptable salt thereof.

Also provided is a method of treating multiple myeloma in a human, the method comprising administering to the human in need thereof:

a) a pharmaceutically effective amount of a proteasome inhibitor, or a pharmaceutically acceptable salt thereof; and
b) a pharmaceutically effective amount of a phospholipase A2 inhibitor, or a pharmaceutically acceptable salt thereof.

Also provided is a method of treating mantle cell lymphoma in a human, the method comprising administering to the human in need thereof:

a) a pharmaceutically effective amount of a proteasome inhibitor, or a pharmaceutically acceptable salt thereof; and
b) a pharmaceutically effective amount of a phospholipase A2 inhibitor, or a pharmaceutically acceptable salt thereof.

Examples of MEK inhibitors useful in the methods herein include trametinib, cobimetinib, binimetinib, selumetinib, CI-1040, and TAK-733.

In the methods herein, trametinib may be administered orally at a daily dose of from about 0.5 mg to about 5 mg. In some embodiments, trametinib is administered orally at a daily dose of from about 0.5 mg to about 2.5 mg.

Cobimetinib may be administered orally in the methods herein at a daily dose of from about 20 mg to about 100 mg. In some embodiments, the daily dose is from about 20 mg to about 80 mg. In other embodiments, the dose for cobimetinib is from about 40 mg to about 60 mg daily.

Binimetinib may be administered orally in the methods herein at from about 15 mg to about 60 mg once or twice daily. In some embodiments, binimetinib is administered at from about 15 mg to about 60 mg twice daily. In some embodiments, binimetinib is administered at from about 30 mg to about 45 mg twice daily.

Selumetinib may be administered in the methods herein at a dose of from about 25 mg to about 100 mg once or twice daily. In some embodiments, the selumetinib dose is from about 25 mg to about 75 mg once or twice daily.

Provided is a method of inhibiting MEK inhibitor-induced autophagy in a human, the method comprising ad ministering to a human in need thereof a pharmaceutically effective amount of a phospholipase A2 inhibitor, or a pharmaceutically acceptable salt thereof.

Also provided is a method of treating ovarian cancer in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating BRAF mutant melanoma in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating NRAS mutant melanoma in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating non-small cell lung cancer in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating breast cancer in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating colorectal cancer in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Also provided is a method of treating melanoma in a human, the method comprising administering to the human in need thereof:

a) a pharmaceutically effective amount of a MEK inhibitor, or a pharmaceutically acceptable salt thereof; and
b) a pharmaceutically effective amount of a phospholipase A2 inhibitor, or a pharmaceutically acceptable salt thereof.

Specific mTOR inhibitors that may be used in the methods herein include Rapamycin, everolimus (Afinitor), temsirolimus (Torisel), sirolimus (Rapamune), Ridaforolimus (AP23573, MK-8669), or deforolimus, zotarolimus, umirolimus, sirolimus NanoCrystal (Elan Pharmaceutical Technologies), sirolimus TransDerm (TransDerm), Sirolimus-PNP (Samyang), biolimus A9 (Biosensors), ridaforolimus (Ariad), TCD-10023 (Terumo), DE-109 (MacuSight)Perceiva (MacuSight), XL-765 (Exelixis), quinacrine (Cleveland BioLabs), PKI-587 (Pfizer), PF-04691502 (Pfizer), GDC-0980 (Genentech and Piramed), dactolisib (Novartis), CC-223 (Celgene), PWT-33567 (Pathway Therapeutics), P7170 (Piramal Life Sciences), LY-3023414 (Eli Lilly), INK-128 (Takeda), GDC-0084 (Genentech), DS-7423 (Daiichi Sankyo), DS-3078 (Daiichi Sankyo, CC-1 15 (Celgene), CBLC-137 (Cleveland Biolabs), AZD-2014 (Astrazeneca), X-480 (Xcovery), X-414 (Xcovery), EC-0371 (Endocyte), VS-5584 (Verastem), PQR-401 (Piqur), PQR-316 (Piqur), PQR-31 1 (Piqur), PQR-309 (Piqur), PF-06465603 (Pfizer), NV-128 (Novagen), nPT-MTOR (Biotica Technology), BC-210 (Biotica Technology), WAY-600 (Biotica Technology), WYE-354 (Biotica Technology), WYE-687 (Biotica Technology), LOR-220 (Lorus Therapeutics), HMPL-518 (Hutchinson China MediTech), GNE-317 (Genentech), EC-0565 (Endocyte), CC-214 (Celgene), ABTL-0812 (Ability Pharmaceuticals), getatolisib, aptiolisib, dactolisib, and sapanisertib.

Provided is a method of inhibiting mTOR inhibitor-induced autophagy in a human, the method comprising administering to a human in need thereof a pharmaceutically effective amount of a phospholipase A2 inhibitor, or a pharmaceutically acceptable salt thereof.

Also provided is a method of treating solid tumors in a human, the method comprising administering to the human in need thereof:

a) a pharmaceutically effective amount of a mTOR inhibitor, or a pharmaceutically acceptable salt thereof; and
b) a pharmaceutically effective amount of a phospholipase A2 inhibitor, or a pharmaceutically acceptable salt thereof.

Also provided is a method of treating non-small cell lung cancer in a human, the method comprising administering to the human in need thereof:

a) a pharmaceutically effective amount of a mTOR inhibitor, or a pharmaceutically acceptable salt thereof; and
b) a pharmaceutically effective amount of a phospholipase A2 inhibitor, or a pharmaceutically acceptable salt thereof.

Also provided is a method of treating glioma in a human, the method comprising administering to the human in need thereof:

a) a pharmaceutically effective amount of a mTOR inhibitor, or a pharmaceutically acceptable salt thereof; and
b) a pharmaceutically effective amount of a phospholipase A2 inhibitor, or a pharmaceutically acceptable salt thereof.

Also provided is a method of treating glioma in a human, the method comprising administering to the human in need thereof:

a) a pharmaceutically effective amount of a mTOR inhibitor, or a pharmaceutically acceptable salt thereof; and
b) a pharmaceutically effective amount of a phospholipase A2 inhibitor, or a pharmaceutically acceptable salt thereof.

Also provided is a method of treating bladder cancer in a human, the method comprising administering to the human in need thereof:

a) a pharmaceutically effective amount of a mTOR inhibitor, or a pharmaceutically acceptable salt thereof; and
b) a pharmaceutically effective amount of a phospholipase A2 inhibitor, or a pharmaceutically acceptable salt thereof.

PI3K inhibitors that may be used in the methods herein include buparlisib, pictilisib, pilaralisib, coplanlisib, afuresertib, alpelisib, apitolisib, dactolisib, duvelisib, idelalisib (ZYDELIG®), ipatasertib, omipalisib, perifosine, pictilisib, sapanisertib, taselisib, and umbralisib. In some embodiments herein the PI3K inhibitor is idelalisib administered once, twice, or three times daily at individual doses of from about 20 mg to about 400 mg. In other embodiments idelalisib is administered once, twice, or three times daily at individual doses of from about 50 mg to about 200 mg. In further embodiments idelalisib is administered once, twice, or three times daily at individual doses of from about 75 mg to about 150 mg.

Provided is a method of inhibiting PI3K inhibitor-induced autophagy in a human, the method comprising ad ministering to a human in need thereof a pharmaceutically effective amount of a phospholipase A2 inhibitor, or a pharmaceutically acceptable salt thereof.

Also provided is a method of treating chronic lymphocytic leukemia in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating relapsed chronic lymphocytic leukemia in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating follicular B-cell non-Hodgkin's lymphoma in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating small lymphocytic lymphoma in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating follicular lymphoma in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating chronic lymphocytic leukemia in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating small lymphocytic leukemia in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating low grade lymphoma in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

Also provided is a method of treating pancreatic ductal adenocarcinoma in a human, the method comprising administering to the human in need thereof:

a) a pharmaceutically effective amount of a PI3K inhibitor, or a pharmaceutically acceptable salt thereof; and
b) a pharmaceutically effective amount of a phospholipase A2 inhibitor, or a pharmaceutically acceptable salt thereof.

### Summary of Experimental Results

To clinically model residual disease in the setting of first-line treatment, we first performed an unbiased screen to identify small molecule inhibitors that increased autophagic flux. We did this because small molecule inhibitors affect multiple targets resulting in both on-target and unanticipated off-target effects, making up-front prediction of drugs that induce autophagic flux challenging. Next, we undertook a comprehensive phosphoproteomic analysis to determine the immediate signaling networks perturbed by initial drug treatment and examined whether these changes impacted metabolic pathways. Finally, we performed global metabolic profiling to systematically document the immediate metabolic adaptations effected by the therapy-induced autophagic processes.

Consistent with clinical findings, we found that despite effective inhibition of growth promoting oncogenic pathways in vitro and in mouse models, residual disease persisted. We show that autophagy-mediated metabolic adaptations supported cancer cell survival. Autophagy was required for these metabolic adaptations because they were abrogated in cells deficient for the essential autophagy gene, ATG5. Subsequently, we identified that PLA2, the rate limiting enzyme responsible for catalyzing the breakdown of phospholipids to lysophospholipids and fatty acids had an important role in the survival of cancer cells in the setting of first-line therapy. Pharmacological inhibition of this enzyme dampened oxidative phosphorylation, and significantly increased apoptosis when combined with first-line treatment. Our findings highlight a previously unappreciated role for PLA2 in conferring a survival advantage to cancer cells in metabolically restricted environments, demonstrate that this enzyme supports autophagy-induced metabolic reprogramming and importantly, can be therapeutically inhibited to override residual disease.

Multiple Small Molecules Induce Autophagic Flux by Inhibiting mTOR

Small molecule inhibitors are the backbone for the treatment of multiple solid and hematological malignancies. However, the natural history of patients on these drugs is one of initial response followed by the maintenance of residual disease, with subsequent emergence of resistance and disease progression [1,2]. We reasoned that the induction of autophagic flux contributes to the maintenance of residual disease. Since it is challenging to precisely identify which cancer drugs induce autophagic flux, we utilized a library of 116 clinically-focused and mechanistically annotated compounds that included activity against two-thirds of the tyrosine kinome as well as other non-tyrosine kinase pathways including MAPKs, PI3K/AKT/mTOR, AMPK, ATM, Aurora kinases, CAMKs, CDKs, GSK3a/b, IkK, PKA, PKC, PLK1, and RAF. We also tested small-molecules with activity against the BCL2 family, BRD4, Hedgehog, HSP90, proteasome, survivin, STAT3, and WNT/beta-catenin [6-8].

We used a metastatic human RCC cell line, ACHN. Small molecule inhibitors have been extensively used in RCC over the last decade. These can be broadly classified as drugs that inhibit the VEGFR tyrosine kinase and those that inhibit mTOR, but neither class of drugs elicit durable clinical responses, making RCC a relevant cancer to study whether treatment-induced autophagy, in its own right, could determine cancer cell survival and thus residual disease [9]. p62 is an adaptor molecule that facilitate the degradation of proteins by autophagy. p62 localizes to the site of autophagosome formation where it recognizes ubiquitinated cargo via its ubiquitin-binding domain (UBA) and delivers it to the autophagosome via its LC3 interaction region (LIR) domain [10]. Importantly, p62 is an autophagy substrate that is degraded along with its cargo, and so is expected to decrease with induction of autophagy [11]. Therefore, we monitored p62 steady-state levels as an initial screen of autophagy flux. Next, we combined the measurement of the p62 with a measurement of mTORC1 activity an independent readout to evaluate autophagy. mTORC1 plays the central role in the anabolic process by increasing protein content through ribosomal biogenesis and elongation of protein translation. Specifically, mTORC1 induces mRNA translation by inducing the activation of p70S6 kinase and the subsequent phosphorylation of the S6 ribosomal protein. To ensure increased cellular biomass, mTORC1 downregulates autophagy [12]. We chose the phosphorylation status of the downstream target of mTORC1 signaling, ribosomal S6 (hereafter S6) as a readout for mTORC1 activity.

To provide a straightforward method for detecting the induction of autophagy, we monitored ACHN cells for small molecule inhibitors which decreased the antibody labelled levels of p62 protein and pS6 phosphorylation (see methods below for detailed experiment procedure). To that end, automated high content imaging (HCl) microscopy was used to detect induction of autophagy as documented by the decrease in autophagy flux by p62 protein expression and mTORC1 inhibition by the decrease in S6 phosphorylation. The HCl assay was optimized for 384-well plate screening. Drug screening plates were designed and created as previously described [6-8]. Heatmap visualization shows division of the test compounds into 5 classes:

(1) reduced expression of both p62 and p-S6;

(2) increased pS6 and p62;

(3) increased pS6 and reduced p62 expression;

(4) increased p62 expression and reduced p-S6 expression; or

(5) minimal or no influence on either marker expression, in dose dependent manner. Compounds that reduced both p62 expression and phosphorylation of S6 are potentially treatment-inducing autophagy drugs. This high priority list of compounds was enriched for inhibitors of mTOR (Rapamycin, PP-242), PI3K (LY-294002, BEZ-235), proteasome (Velcade) and inhibitors which non-specifically inhibits multiple kinases such as Flavopiridol, Staurosporine and H89. Therefore, data from these assays confirmed many of the known mechanisms of actions of the library panel, e.g. mTOR inhibitors inducing autophagic flux, confirming the validity of the HCl screen. Notably, the HCl screen also identified several compounds that would not have been predicted to induce autophagy such as a tankyrase kinase inhibitor (XAV939) and an aurora kinase inhibitor (KW2449).

Remarkably, the screen identified several structurally different Janus kinase (JAK) inhibitors as potent inducers of autophagic flux, namely, pan-Jak inhibitor (Jak 1, 2, 3), Go6978 (Jak 2), ruxolitinib (Jak 1, 2) and CYT387 (Jak 1, 2). All four drugs potently inhibited S6 phosphorylation, pointing to a mTORC1-dependent mechanism. Ruxolitinib (Jakafi®) is FDA-approved for myeloproliferative neoplasms (MPN) [13, 14]. MPN is usually associated with JAK2 V617F mutations, which was the basis for the development of JAK. CYT387 (momelotinib) is an orally available Jak 1-2 inhibitor that has improved splenomegaly and reduced anemia in MPN patients [14, 15]. Based on its clinical efficacy, CYT387 is currently being evaluated against ruxolitinib as first-line treatment for intermediate to high risk MPN patients. We observed that CYT387 was effective in suppressing the phosphorylation of JAK and STAT3 in multiple human RCC and MPN cell lines. In addition, we noted that CEP-701 and TG-101384, two additional JAK inhibitor not contained in our library also potently induced autophagy and inhibited mTORC1. Recently, ruxolitinib has been reported to induce autophagy in leukemia cells [16]. Since JAK inhibitors as a class of compounds scored highly in our screen, and because CYT387 was the most potent JAK inhibitor to induce autophagic flux and simultaneously decrease p-S6 phosphorylation in solid tumor cells in our HCl screen, we selected this small molecule for further validation.

The Janus Kinases (JAKs) phosphorylate signal transducers and activators of transcription (STAT) transcription factors on tyrosine-705 resulting in dimerization and nuclear translocation and subsequent activation of proliferative and anti-apoptotic target genes [17]. Recently it has been shown that cytoplasmic, non-phosphorylated STAT3 inhibits starvation-induced autophagy, in an mTOR-independent manner [18]. However, in contrast to these findings, the JAK inhibitors identified in our screen induce autophagy in an mTORC1 dependent fashion. Several controls supported our findings that CYT387 induced autophagic flux by inhibiting mTORC1. First, depletion of JAK or STAT3 by siRNA did not induce increase autophagy (as indicated by baseline lipidation of LC3) and did not decrease the phosphorylation of S6. Second, knockdown of JAK or STAT3 did not increase the conversion of LC3-I into LC3-II by CYT387. Together these results indicate that the JAK induced autophagy seen in our RCC cells are not due to the inhibition of cytoplasmic STAT3. We further validated our findings with the following experiments in multiple RCC and MPN cell lines and in clinical patient tumors (details below). Interestingly, we observed that treatment with CYT387 in multiple human RCC and MPN cell lines was primarily cytostatic, and suggests that treatment-induced autophagy promotes cancer cell survival.

To confirm the original HCl results, we plated ACHN cells on coverslips, treated with CYT387 and stained for p62 and p-S6. CYT387 treatment resulted in decreased p62 protein expression and phosphorylated-S6 levels by immunofluorescence staining. Immunoblots confirmed the induction of autophagy by CYT387 as seen by the conversion of LC3-I to LC3-II, the degradation of p62 and inhibition of mTORC1 (as seen by decrease in phosphorylated S6). CYT387 decreased STAT signaling as seen by the decrease in phosphorylation of Y705-STAT3. Next, we stably expressed a mChery-EGFP-LC3 reported in ACHN cells. This reporter for autophagic flux takes advantage of the fact that EGFP fluorescence is quenched in the acidic environment of the autolysosome relative to mCherry [19]. CYT387 treatment resulted in decreased expression of green-yellow cells and increased expression of red cells. This method to measure flux has been extensively validated and accurately quantitates autophagic flux induction by multiple stimuli and chemical and genetic inhibition of autophagy. We also stained CYT387 treated ACHN cells with the autofluorescent compound monodansylcadaverine (MDC), which acts as a lysosomotropic agent and labels some of the acidic compartments that are observed after fusion with lysosomes (autolysosomes), and found that CYT387 increased MDC autoflorescence in ACHN cells, consistent with the induction of autophagy [20]. LC3-I is normally converted into LC3-II (LC3-I covalently bound to phosphatidylethanolamine) during autophagosome formation but is converted back to LC3-I by protease cleavage during autophagosome maturation. The overall formation of LC3-II was detected by preincubating cells with E64d/pepstatin, inhibiting protease-induced reconversion of LC3-II into LC3-I. CYT387 increased LC3-II levels in ACHN cells, and this increase was more pronounced in the presence of E64D/pepstatin, consistent with an increase in autophagosome formation. CYT387 was able to induce autophagy in a dose dependent manner in mouse embryo fibroblasts (MEFs) that retained the essential autophagy gene, Atg5 (Atg5+/+), as seen by the lipidation of LC3 [21, 22]. Conversely, CYT387 did not induce autophagy in Atg5 deficient cells (Atg5−/−). Likewise, CYT387-induced autophagy was abrogated with siRNA depletion of Atg5 in ACHN cells. Taken together, these results indicate that CYT387 treatment induces autophagy flux in human RCC cells.

CYT387 increased the number of double-membraned autophagosomes, which are pathognomonic of autophagy (as determined by transmission electron microscopy, TEM) [23]. Interestingly, this was accompanied by increased number of mitochondria and lipid droplets (see below). Transcriptomic analysis of CYT387 treated ACHN cells using gene set enrichment (GSEA) of multiple independent datasets revealed significant enrichment of genes involved in several metabolic pathways, e.g. lysosome activity, peroxisome activity, PPAR signaling, arachidonic acid, sphingolipid and fatty acid metabolism. GSEA of an independent autophagy-lysosome gene dataset confirmed that CYT387 treatment increased the expression of autophagy-lysosome genes (NES: 2.06, p=0.006) [24]. Additionally, we observed enrichment for a PGC1A gene signature suggestive of increased mitochondrial biogenesis (NES: 1.73, p=0.0201) [25].

Genesets related to inhibition of cell cycle, ribosome activity, protein and pyrimidine metabolism were significantly downregulated by CYT387. Specifically, biological modules associated with mTOR (e.g. cell cycle, protein synthesis) were also anti-correlated with CYT387 treatment. CYT387 treatment downregulated genes involved in glycolysis such as PFKB3 and HK2, and the upregulated of negative regulators of pyruvate metabolism including PDK4 and PDK2, consistent with the fact that mTORC1 promotes glycolysis [26,27]. Together, these data support the observation that CYT387 treatment confers a selective repression of transcriptional networks induced by mTOR.

To extend our studies into clinical samples, we exposed patient-derived RCC organotypic cultures to CYT387 treatment for 24 hours. Importantly, CYT387 significantly induced LCB expression while simultaneously reducing phosphorylated S6 levels, consistent with our preclinical finding that CYT387 induces autophagy by inhibiting mTORC1. Collectively, we describe a two-step high content phenotypic screen that allows the identification of small molecule inhibitors that induce autophagic flux in preclinical and clinical samples, with particular emphasis on mTORC1 inhibition as a mechanism of action. In line with our reasoning that predicting drugs that induce autophagy would be difficult, we unexpectedly uncovered a class of JAK inhibitors which induced autophagic flux in human RCC and MPN cancer cells. Moreover, our data indicate early engagement of distinct transcriptional and metabolic adaptations to treatment.

Treatment-Enforced Signaling is Coupled with Changes in Metabolic Pathways

To obtain further insight into the signaling pathways affected by CYT387 treatment, we studied changes in the phosphoproteome of two different human RCC cells (ACHN and SN12C) after CYT387 treatment, using a simplex tandem mass tag (TMT) technology [28-30].

Of the 1,896 phosphoserine and phosphothreonine peptides (pST) and 640 phosphotyrosine peptides (pY) identified, supervised hierarchical clustering revealed 513 pST peptides and 180 pY peptides significantly differed between treated and untreated cells. Quantifying the phosphoproteomic data we observed several phosphopeptides to predict mTORC1 suppression Tuberous sclerosis complex 2 (TSC2) in CYT387-treated cells are hypophosphorylated at two inhibitory phosphoresidues, T¹⁴⁶² [31]) and S¹⁷⁹⁸ [32], suggesting that CYT387 treatment enhances TSC2 activity. TSC2 suppresses mTORC1 activity by converting Rheb into its inactive, GDP-bound state [33]. Rapamycin-insensitive companion of mTOR (RICTOR) in CYT387-treated cells is hypophosphorylated at T¹¹³⁵. RICTOR is a subunit of mTOR complex 2 (mTORC2) [34], but the phosphorylation of T¹¹³⁵ is mediated by mTORC1 via induction of p70S6 kinase [35] and impedes the ability of mTORC2 to phosphorylate AKT on S⁴⁷³ [36]. This suggests that CYT387 treatment increases the activity of mTORC2. As expected, ribosomal protein S6 (RPS6) trended towards hypophosphorylation in CYT387-treated cells. Sequential phosphorylation on RPS6 enhances its ability to associate with the m7GpppG cap, enhancing translation initiation [37]. CYT387 treatment also trended towards hypophosphorylation of STAT3 Y⁷⁰⁵, as expected, and hyperphosphorylation of the insulin receptor (INSR) at Y¹¹⁸⁹, a phosphoresidue that induces activity [38]. Though the exact relationship between INSR Y¹¹⁸⁹ and mTOR is unclear, INSR is activated by mTORC2 through tyrosine kinase activity [39].

To expand upon the kinases that may be perturbed or activated upon CYT387 treatment, we performed kinase-substrate enrichment analysis (KSEA) to examine inferred differential kinase activity [40]. As expected, we found that p70S6 kinase (RPS6 KB) is significantly less active in CYT387-treated cells. Interestingly, glycogen synthase kinase 3 beta (GSK3B) is also less active after CYT387 treatment.

However, inferred activity of AKT is inconclusive as some motifs trend toward increased activity and others trend toward decreased activity in CYT387-treated cells. In addition, we generated a gene list that is relatively more active in CYT387-treated cells based on our phosphoproteomic data. Performing DAVID analysis on this gene list revealed several KEGG pathways that are biologically relevant to CYT387 treatment, including insulin signaling, glycolysis, amino acid biosynthesis, and central carbon metabolism [41,42]. In all, the phosphoproteome provides strong evidence that CYT387 treatment reduces mTORC1 signaling to increase mTORC2 signaling leading to AKT activation. Notably, CYT387-induced kinome reprogramming is coupled with changes in metabolic pathways.

Both our preclinical and clinical experiments indicate that CYT387 inhibits mTORC1, as indicated by the decrease in S6 phosphorylation. However, we suspected that the CYT387-induced inhibition of mTORC1 would relieve the inhibitory feedback signal normally transmitted from mTORC1 to PI3K as the phosphoproteomic data suggested via KSEA analysis. This would then hyperactive PI3K signaling. Consistent with this interpretation, CYT387 treatment caused an increase in AKT T308, the PDK-1 catalyzed site that serves as readout for PI3K signaling in a time-dependent manner. CYT387 induces autophagy in ACHN cells which is rapidly reversible, as seen by the reduction in LC3B lipidation within 24 hrs of removal of drug, and correlated with reversal of the p-STAT3, p-S6, p-AKT phosphorylation patterns. This is in line with treatment-induced autophagy mediating survival in the setting of oncogenic signaling inhibition with return to growth when the stressor is removed. Further, we saw no effect on the phosphorylation of ERK 1/2).

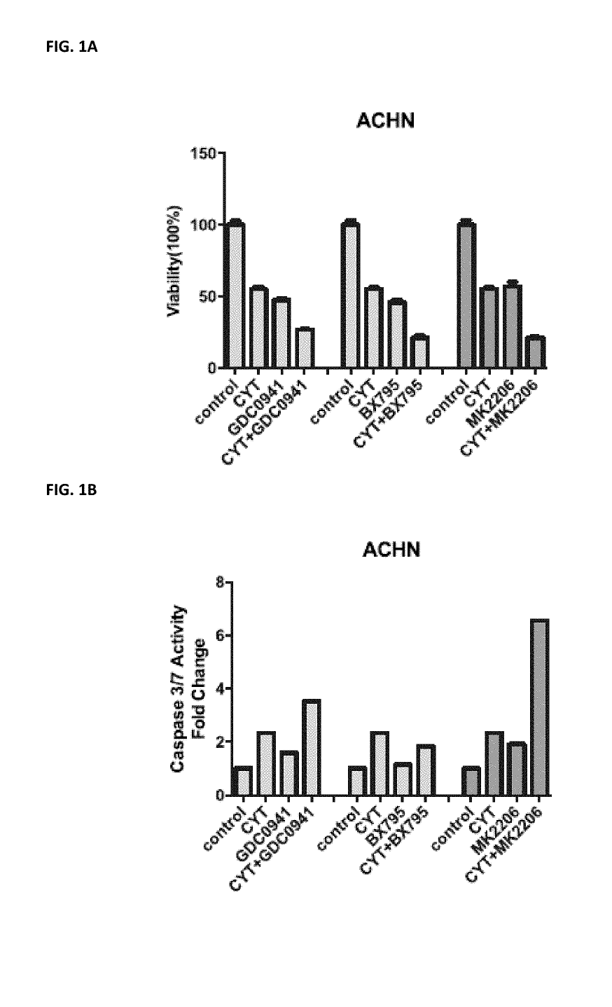

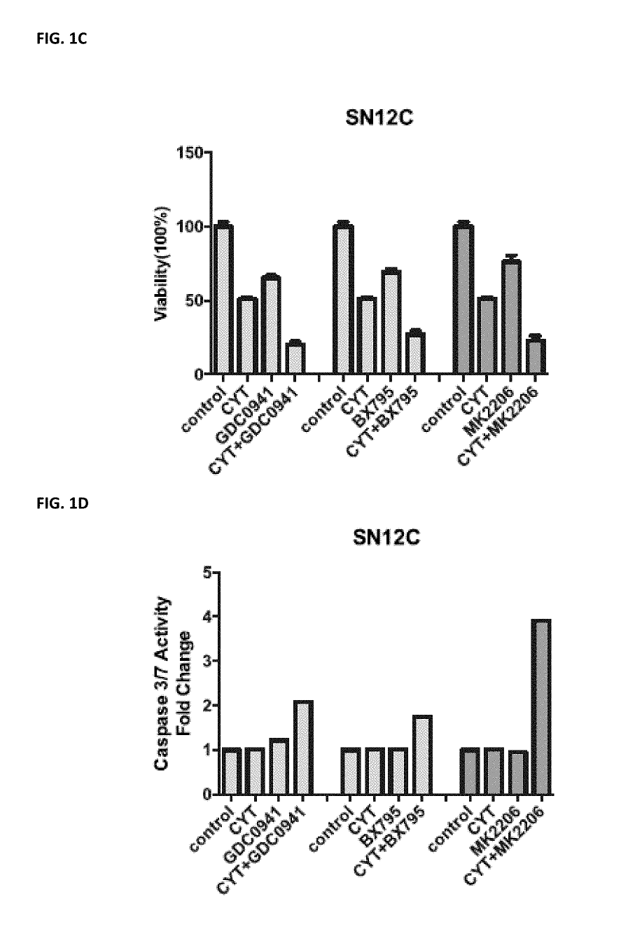

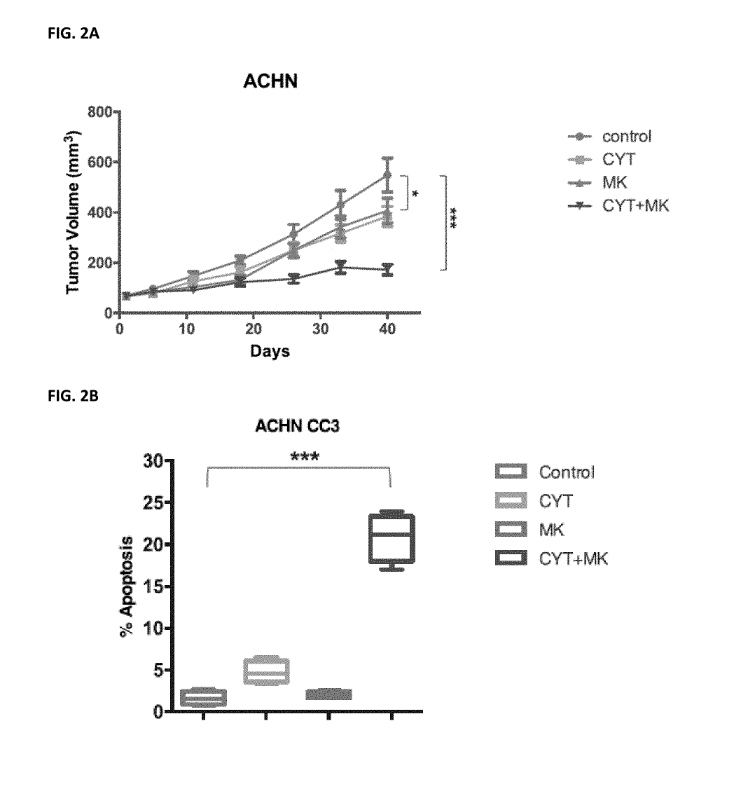

This activation of PI3K-AKT that results from CYT387 induced inhibition of mTORC1 may contribute to the cytostatic effects seen with CYT387 treatment because AKT promotes cell survival [43]. Therefore, we sought to identify PI3K-AKT pathway inhibitors that would effectively cooperate with CYT387 to induce apoptosis. We used GDC-0941, a pan-PI3K inhibitor [44]; BX795, a PDK-1 inhibitor [45], and MK2206 [46], an allosteric AKT inhibitor to chemically deconstruct this signaling pathway. Cartoon depicts the combination strategy. We first assessed the biologic effects of these inhibitors on proliferation and apoptosis in human RCC cells, singly and in combination with CYT387. While GDC-0941, BX795, MK2206 alone exhibited some anti-proliferative effects, the combination with CYT387 resulted in significantly greater inhibition of proliferation in ACHN and SN12C cells. In marked contrast, all drugs as single agent had little or no effect on apoptosis, but the combination of either agent with CYT387 resulted in increased apoptosis. This was most striking in the CYT387 and MK2206 combination (FIG. 1A, 1B, 1C, 1D). Pharmacodynamics studies demonstrated that the CYT387+GDC-0941 and CYT387+MK2206 combination potently inhibited PI3K, mTORC1 and mTORC2 signaling as monitored by AKT T308, S473 and S6 phosphorylation, respectively which correlated with the increase in caspase 3/7 activity in vitro. We also observed that GDC-0941 or MK2206 had an additive inhibitory effect on mTORC1 activity in cells treated with CYT387, suggesting that the combination may affect mTORC1 by also inhibiting a parallel pathway. In contrast, BX795 despite inhibiting the PDK1 mediated phosphorylation of AKT308 was not able to inhibit mTORC1 or mTORC2 signaling (as monitored by AKT S473 and S6 phosphorylation), which may contribute to its minimal effect on eliciting apoptosis.

We observed that autophagy was induced as determined by the conversion of LC3-I to LC3-II in the co-treated cells. Next, we extended these results to MPN cells, where CYT387-MK2206 combination also induced apoptosis, as evidence by the increase in cleaved caspase 3. Importantly, the CYT387-MK2206 combination induced autophagy in patient derived organotypic RCC cultures.

To further define the role of treatment induced-autophagy in mediating survival, we assessed the effects of CYT387 and MK2206 combination treatment on Atg5−/− and Atg5+/+MEFs. The CYT387-MK2206 co-treatment induced more apoptosis in Atg5−/− MEFS than it did in wild-type controls (demonstrated by increase in cleaved-caspase3) indicating that autophagy protects cells from apoptosis. This suggests that despite effective inhibition of PI3K-AKT-mTOR signaling with resultant induction of apoptosis, RCC cancer cells are able to simultaneously induce an autophagic-fueled survival pathway.

In aggregate these results suggest that overriding the CYT387 mediated activation of PI3K-AKT signaling would effectively suppress the growth of human cancer cells by inducing apoptosis. However, since GDC-0941 co-treatment with CYT387 did not induce apoptosis as effectively as MK2206, and due to the clinical toxicities associated with pan-PI3K inhibitors, we selected MK2206 for further in vivo studies.

### In Vivo First-Line Treatment Restrains Tumor Growth but Residual Disease Persists

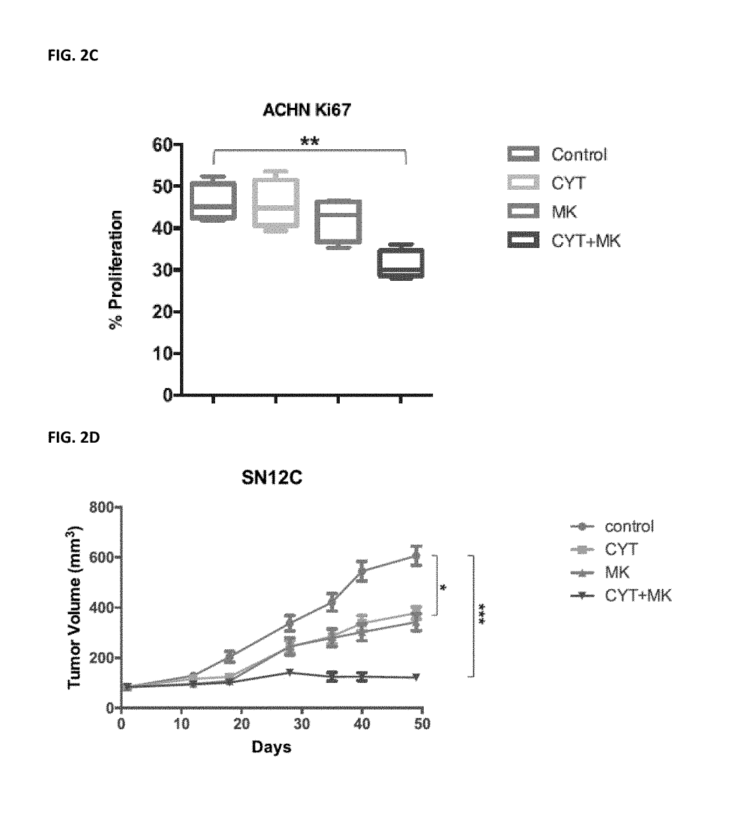

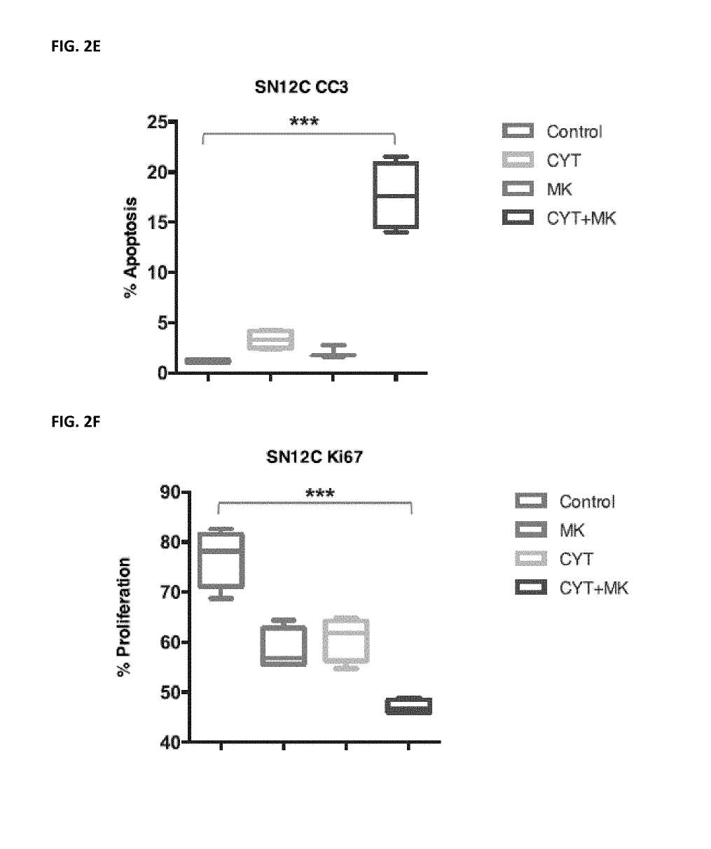

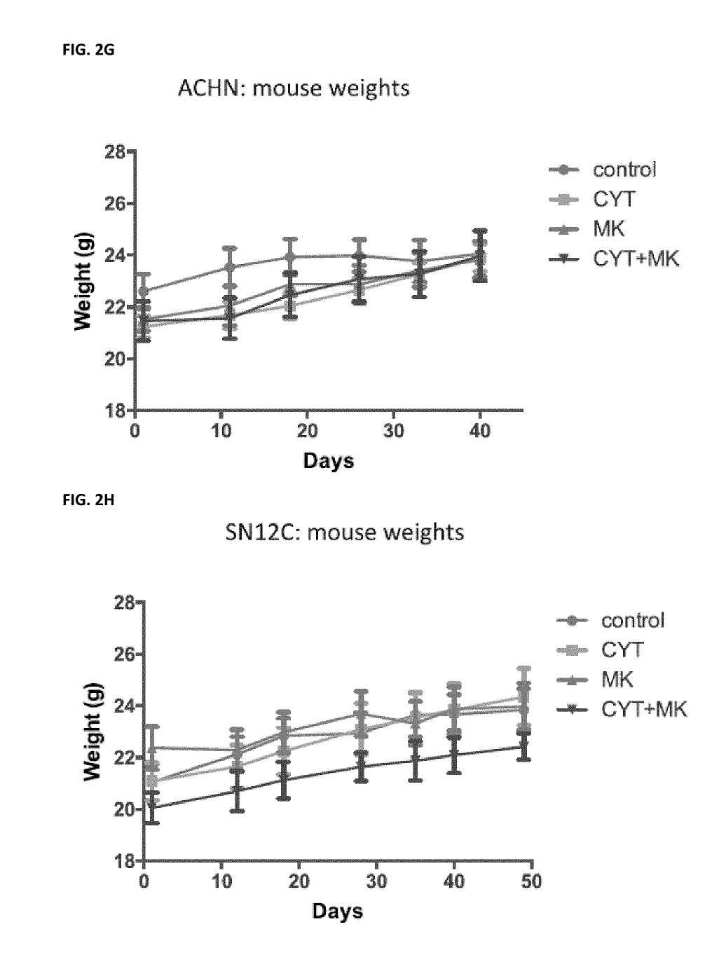

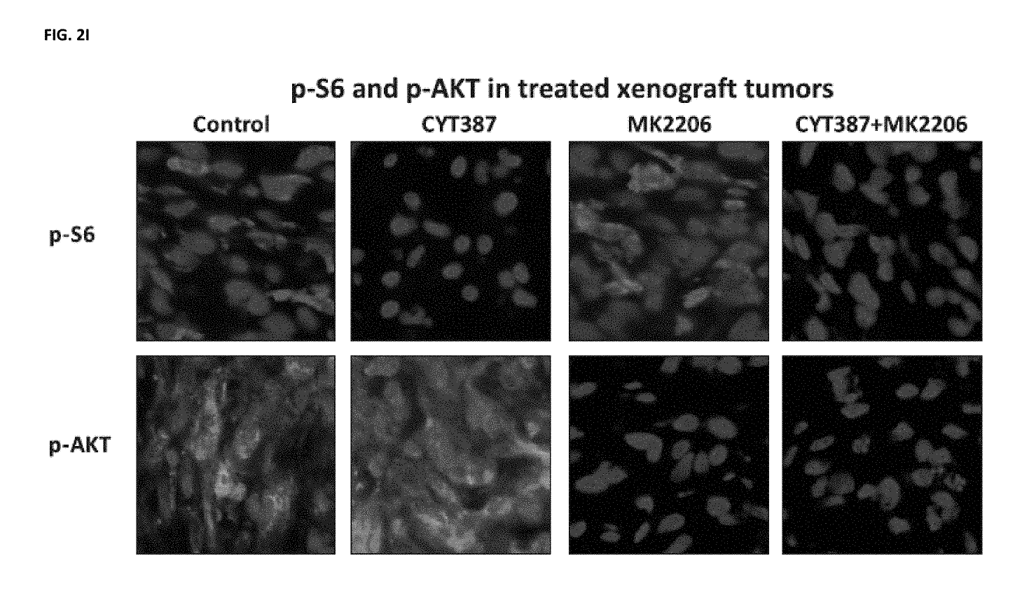

We next examined the safety and efficacy of CYT387 and MK2206 co-treatment in vivo in two xenograft tumor models. While CYT387 or MK2206 alone exhibited antitumor effect on ACHN and SN12C xenografts, the combination of CYT387 with MK2206 resulted in significantly greater tumor growth inhibition (78% TGI) in ACHN and (93% TGI) in SN12C tumor xenografts (p<0.001; Figure, respectively) (FIG. 2A, 2D). Importantly, combination treatment was well tolerated, with no weight loss recorded (FIG. 2G, 2H). Pharmacodynamic studies demonstrated that combination therapy led to the suppression of S6 and AKTS473 phosphorylation (FIG. 2I). Consistent with our in vitro finding, CYT387 alone had minimal impact on apoptosis. In marked contrast, combination treatment with CYT387 and SN12C resulted in significant increase in apoptosis (established by increase in cleaved-caspase3, p<0.001; FIG. 2B: ACHN xenograft tumors; FIG. 2E: SN12C xenograft tumors) and reduction in proliferation (demonstrated by decrease in Ki-67, p<0.001; FIG. 2C: ACHN xenograft tumors; FIG. 2F: SN12C xenograft tumors).

Therefore, prolonged inhibition of PI3K-AKT-mTOR signaling by combining CYT387 and MK2206 is associated with enhanced suppression of tumor growth with good tolerability. However, our findings recapitulate the clinical setting, where despite effective first-line inhibition of an essential growth and survival pathway, residual disease persists.

### Treatment-Induced Metabolic Reprogramming is Supported by Redox Homeostasis

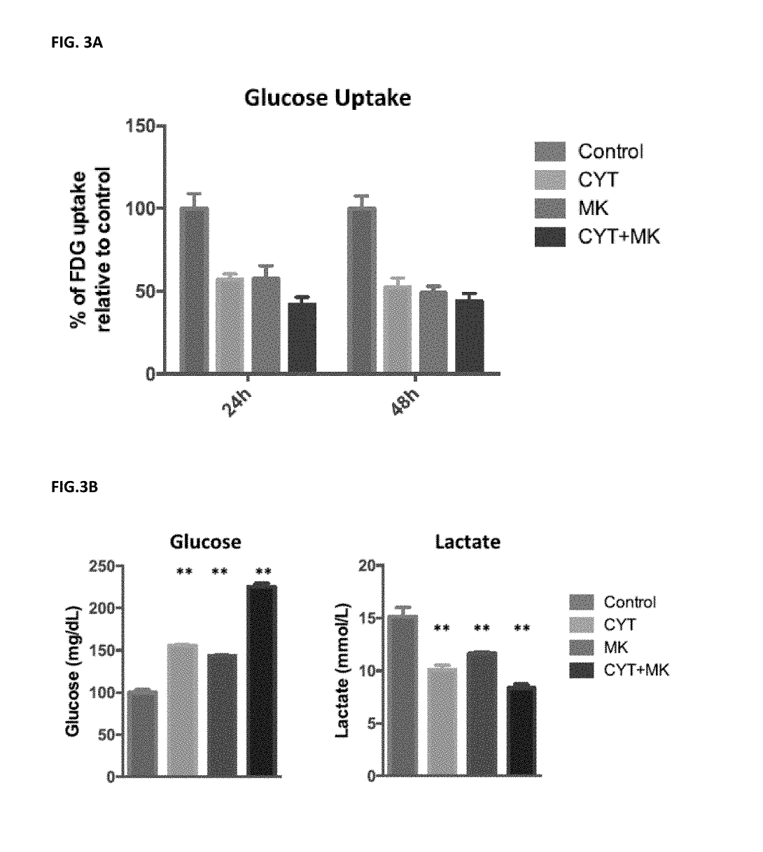

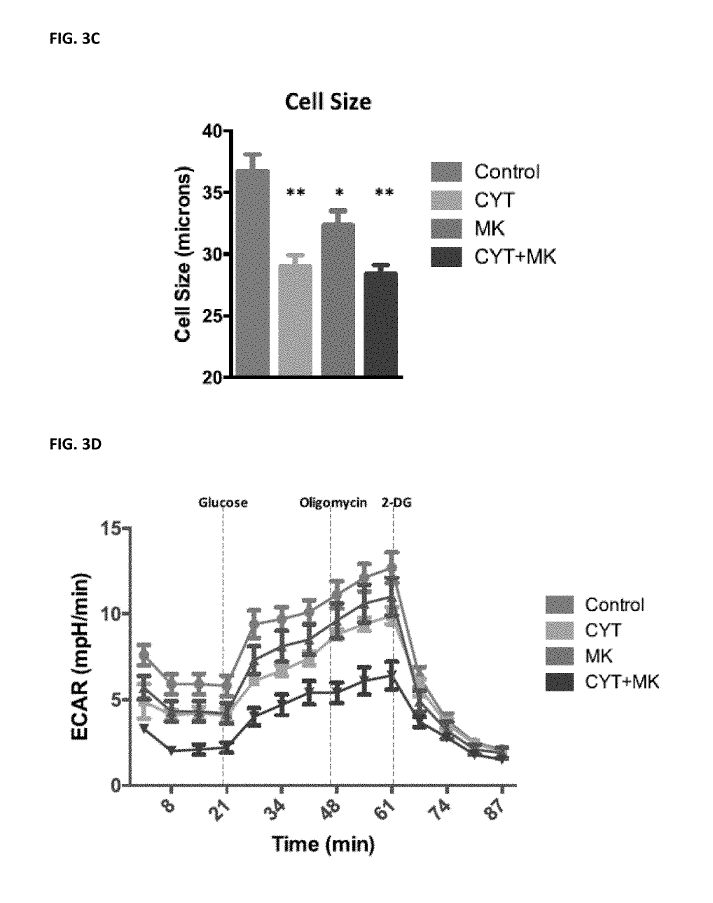

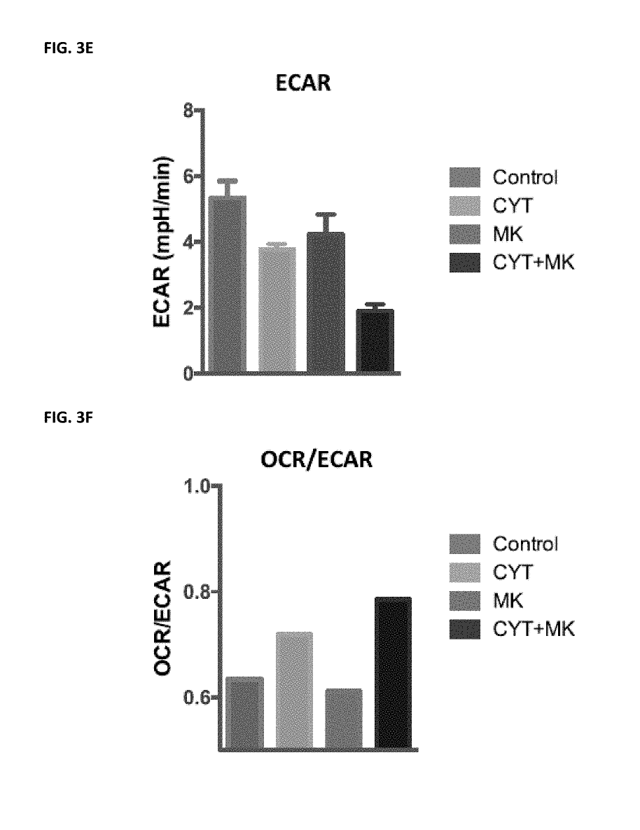

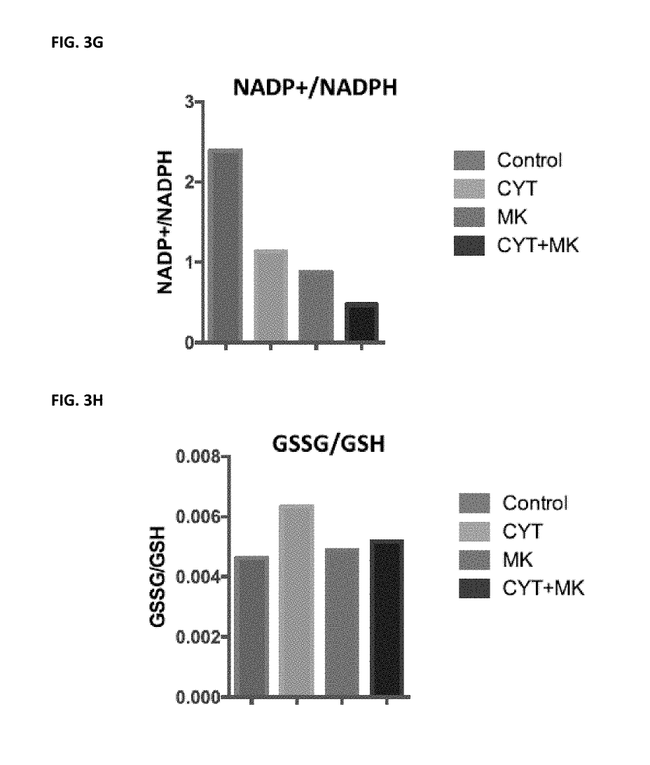

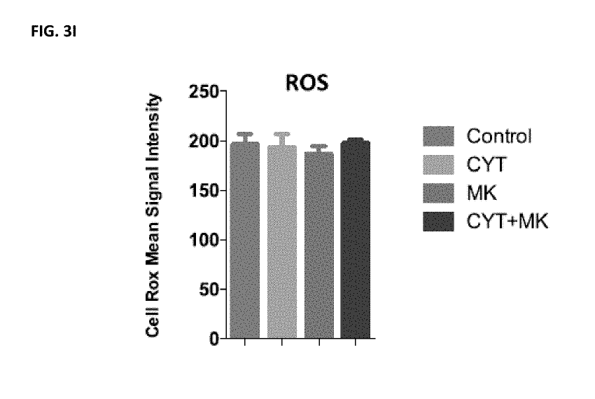

The PI3K-AKT-mTOR pathway regulates multiple steps in glucose uptake and metabolism [26]. Therefore, we hypothesized that CYT387 and MK2206 treatment singly, and in combination would negatively impact glucose uptake, aerobic glycolysis and subsequently biosynthetic pathways, resulting in a glucose-limiting microenvironment. To determine the contribution of CYT387 and MK2206 treatment on the regulation of glycolysis, we measured glucose uptake, lactate excretion and the extracellular acidification rate (ECAR) as readouts for glycolysis. CYT387, MK2206 and the combination significantly decreased glucose uptake and reduced lactate production in vitro (FIG. 3A, 3B). The dramatic difference between lactate/glucose ratio in extracellular media further supports the finding that CYT387 and MK2206 co-treatment inhibits glycolysis (Control: 1.51; CYT387:0.65; MK2206: 0.81; CYT387+MK2206: 0.37) This impaired carbon metabolism with treatment also resulted in reduction of cell size (FIG. 3C). Consistent with the above finding, CYT387 MK2206, and the CYT387-MK2206 combination significantly reduced the ECAR (FIG. 3D, 3E).

Decreased glucose availability with co-treatment might also be reflected in changes with oxidative phosphorylation (OXPHOS) activity, as measured by O2 consumption rate (OCR, an indicator of OXPHOS). However, we found that the OCR/ECAR ratio increased after co-treatment, suggesting a predominant decrease in glycolysis with maintenance of mitochondria-driven OXPHOS (FIG. 3F). Consistent with glucose limitation and decreased glycolysis, we observed increased AMPK phosphorylation at Thr-172, an established indicator of metabolic stress. Importantly, in the setting of glucose deprivation and impairment of the PPP, AMPK has been shown to increase NADPH levels from increased fatty acid oxidation. Specifically, we noted increased levels of NADPH, maintenance of GSSG/GSH ratios and resultant mitigation of reactive oxygen species (ROS) (FIG. 3G, 3H, 3I). These findings are consistent with the role of AMPK in mitigating metabolic stress and promoting cancer cell survival [47]. In comparison, we did not see any reduction in PKM2 levels, suggesting that the metabolic switch from aerobic glycolysis to oxidative phosphorylation is not dependent on pyruvate kinase activity [48].

Overall, these findings suggest that CYT387-MK2206 co-treatment by inducing a glucose-depleted microenvironment severely reduces the glycolytic capacity needed to supply the bioenergetics needs of the RCC cells. Importantly, this treatment-induced nutrient depleted condition, while suppressing proliferation simultaneously promotes survival by regulating NADPH homeostasis and maintaining mitochondrial-driven oxidation.

### Treatment-Induced Autophagy Sustains Residual Disease by Metabolizing Phospholipids

Therefore, to comprehensively determine how autophagy contributes to the metabolic needs, we performed global metabolic analysis using liquid chromatography coupled with tandem mass spectrometry (LC-MS/MS) based platform [49]. These studies revealed that CYT and MK2206, singly and in combination effected changes across multiple pathways. Consistent with the role of the PI3K-AKT-mTOR pathway in the regulation of glycolysis, treatment with these agents was accompanied by reductions in glucose, glucose-6-phosphate, DG3P, PEP, pyruvate and lactate, consistent with the inhibition of glycolysis, as described above and also concordant with the gene expression data. Similarly, we also observed reductions in pentose phosphate pathway intermediates, amino acids, TCA cycle intermediates, ribose biosynthesis and corresponding increases purine breakdown products guanine and hypoxanthine. These findings are in keeping with a nutrient-deprived state (i.e. decreased anabolism) with subsequent increased autophagic catabolism to maintain survival [50]. Cells adapt to glucose deprivation by subsisting on fatty acids, mobilized through glycerolipid remodeling, for oxidation and this is consistent with our observation that the most significant metabolite changes were in lipid intermediates including phospholipids, triacylglycerol (TAG), cholesterol esters, diacylglycerol (DAG) and fatty acids (C16:0, C18:0, C18:1) [51-53].

We further investigated the lipid substrates that were catabolized by autophagy to produce fatty acids for fatty acid oxidation. Steady state metabolite profiling showed significant increases in lysophospholipids and arachidonic acid with corresponding decreases in their phospholipid precursors. Phospholipids, which include phosphatidylcholine (PC), phosphatidylethanolamine (PE), phosphatidylserine (PS), phosphatidylglycerol (PG) and phosphatidylinositol (PI), are major structural components of cellular membranes. The general structure of phospholipids is that of a three-carbon glycerol molecule with a fatty acyl or alkyl group at the sn-1 and fatty-acyl groups at the sn-2 positions; a phosphate moiety and polar head group occupy the sn-3 position and give the phospholipids their amphipathic character necessary for forming bilayer structure. Phospholipase A2 (PLA2) is the enzyme that catalyze the hydrolysis of the phospholipid sn-2 ester bond with subsequent release of lysophospholipids e.g. lysophosphatidylcholine (LPC), alkyl-lysophosphatidylcholine (alkyl-LPC), and free fatty acids [54]. Accordingly, we found elevated levels of C16:0 LPC, C18:0 LPC, C18:1 LPC, C18-0 alkyl LPC and corresponding decreases in their phospholipid precursors. Notably, we observed significant decreases in free fatty acids (C16:0, C18:0, C18:1), supporting the idea that phospholipids are hydrolyzed to supply fatty acids for fatty acid oxidation.

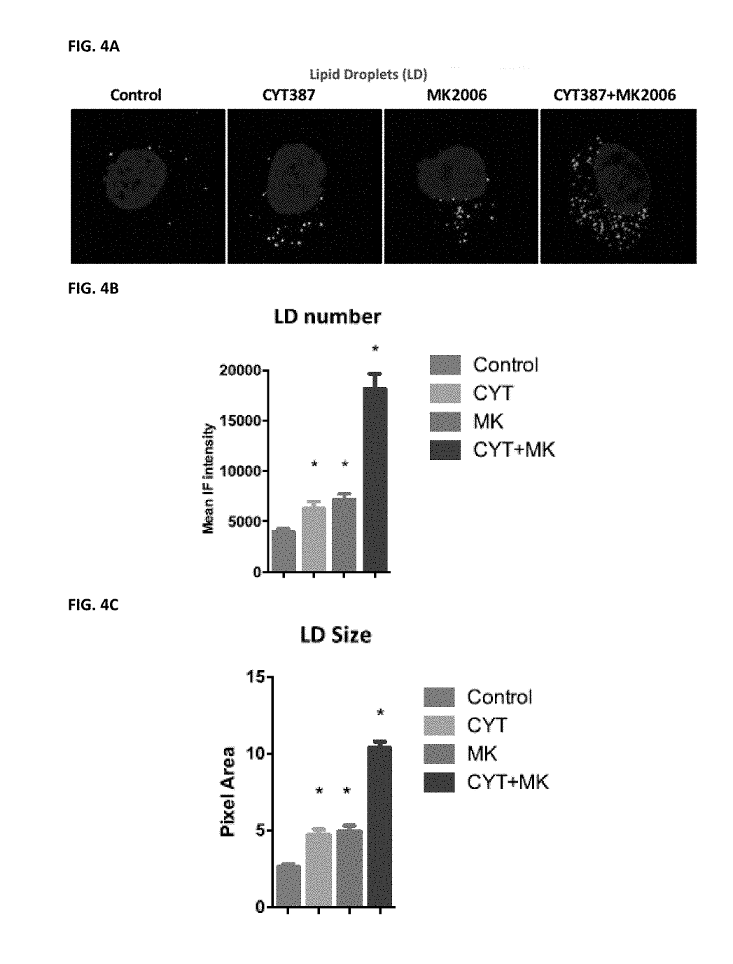

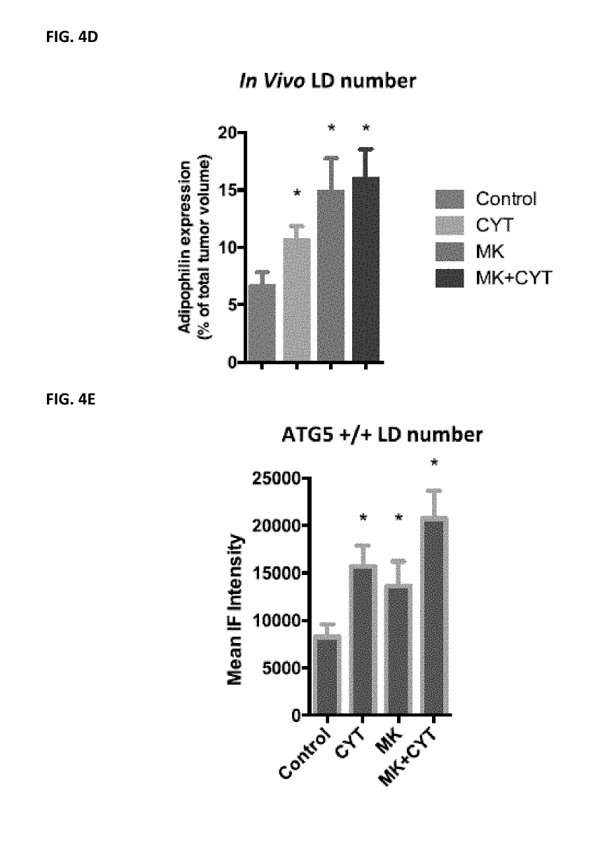

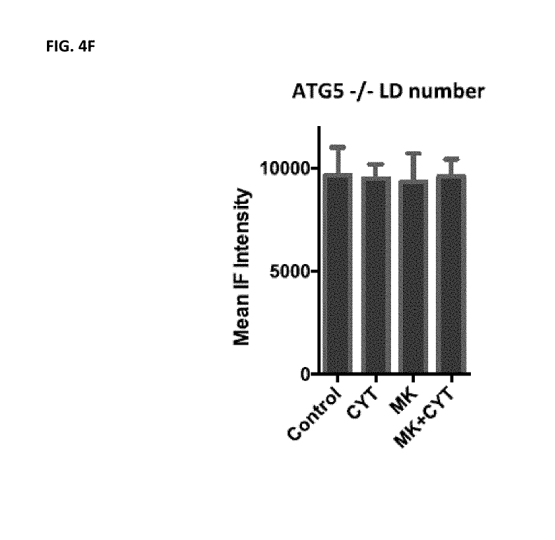

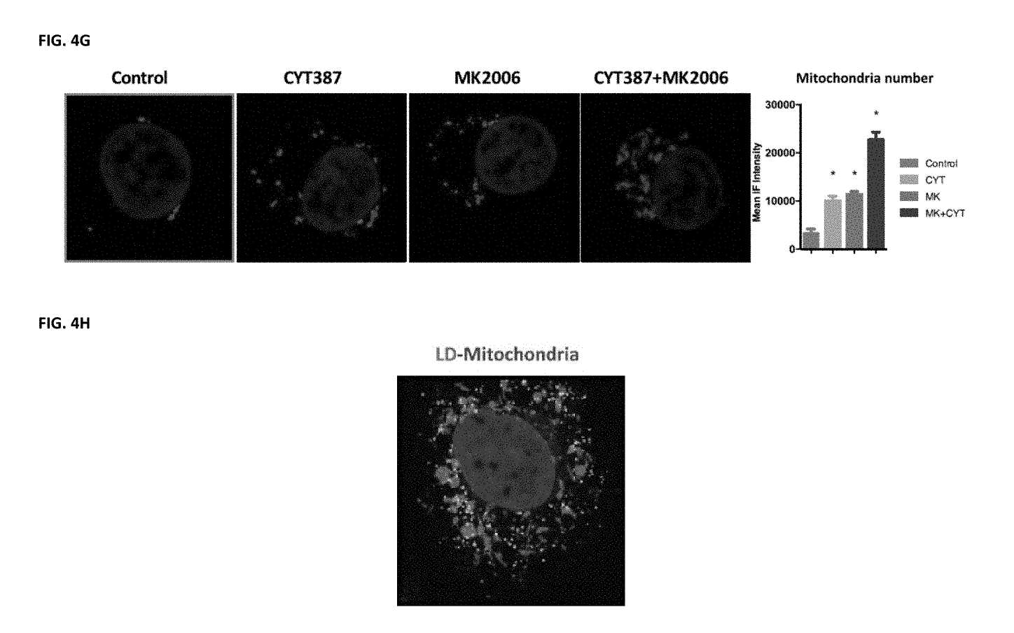

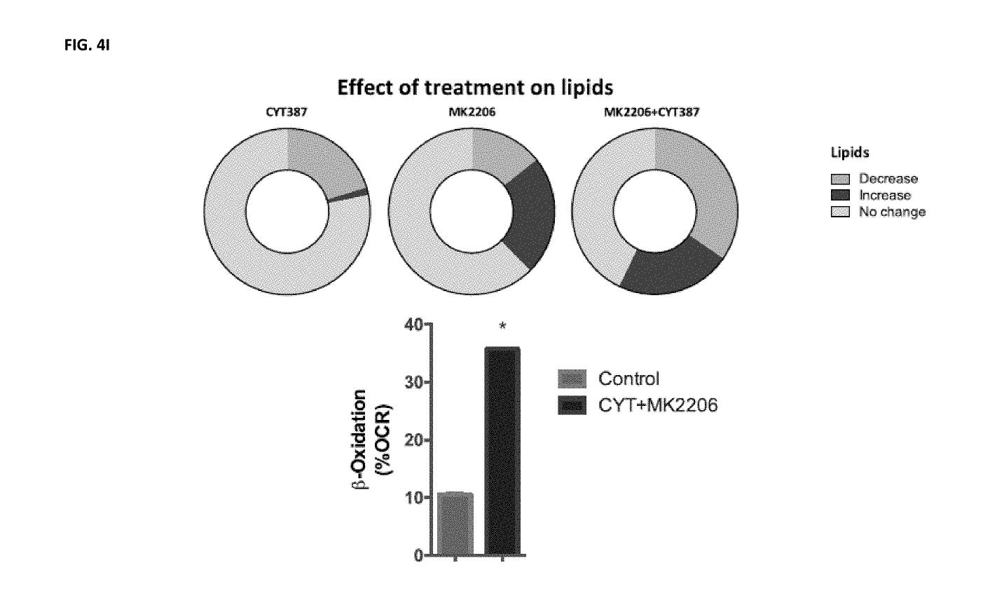

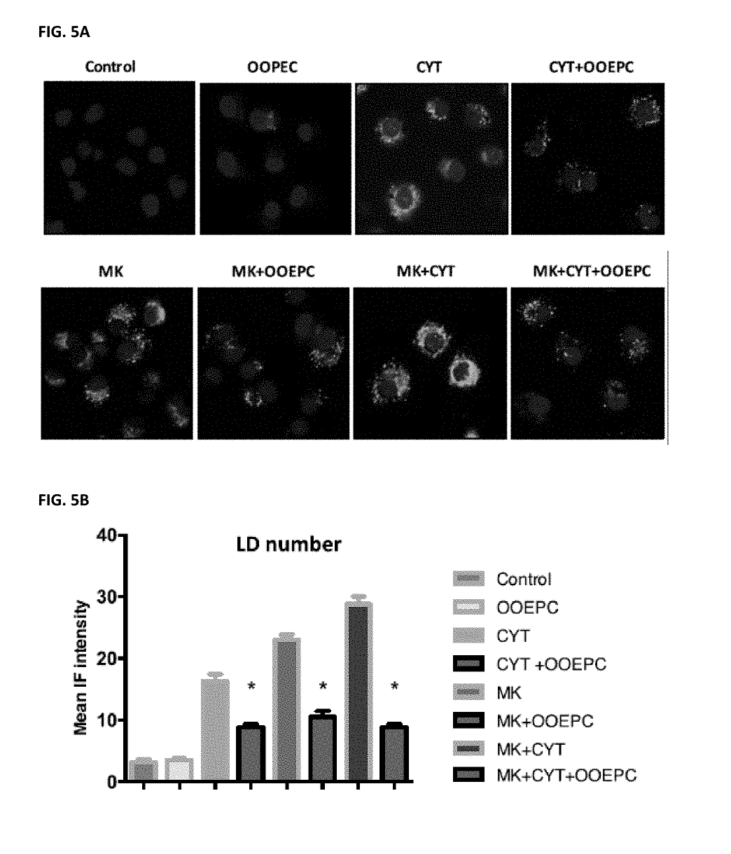

To protect cells from the destabilizing effects of excess lipids, free fatty acids mobilized by autophagy and destined for oxidation are stored in an intermediate intracellular pool, lipid droplets (LDs) [55]. We reasoned that the large changes in glycerolipid redistribution identified by our metabolomics profiling of treated cells would result in increased number of LDs to support fatty acid oxidation, with subsequent mobilization of fatty acids to mitochondria under these nutrient depleted conditions [56]. Consistent with this, we observed that CYT387, MK2206 singly and in combination incrementally and significantly increased the number and size of BODIPY (493/503, green)-labeled LD (FIG. 4A, 4B, 4C).

To determine whether the increase in LDs occurred in vivo, we stained the vehicle, CYT387, MK2206 and CYT387-MK2206 co-treated xenograft tumors for adipophilin, which belongs to the perilipin family, members of which coat intracellular lipid storage droplets and facilitate metabolic interactions with mitochondria [57]. Consistent with the in vitro data, the number of adipophilin-positive LDs significantly and incrementally increased with treatment (as measured on treatment day 40 in ACHN xenograft tumors; CYT387<MK2206<CYT387+MK2206; p=0.0046) (FIG. 4D), indicating that these drug treatments stimulate the formation of lipid droplets in vivo. Collectively, this data suggests that the early adaptive and survival changes effected by the initial drug treatment continues to support maintenance of long-term in vivo tumor growth.

Next, to further determine if autophagy contributed to LD numbers, we treated Atg5+/+ and Atg5−/− MEFs with CYT387, MK2206 and the combination. Autophagy competent Atg5+/+MEFs were able to significantly increase LD numbers (FIG. 4E). In marked contrast, none of the treatment regimens were able to increase LDs in Atg5−/− MEFs, confirming that autophagy is required to sustain LD levels (FIG. 4F). This is in line with a model where autophagy of cellular organelles and membranes during nutrient deprivation produces fatty acids that supply the LD pool, where they are then transferred into mitochondria for β-oxidation. In support of this, we observed that treated RCC cells had significantly increased number of mitochondria (FIG. 4G). Accordingly, dual staining of treated ACHN cells with a mitochondrial marker (Mitotracker-orange) and LDs with Bodipy (green) revealed that the LDs were closely associated with the mitochondria, potentially enabling the fatty acids released from lipid droplets to traffic directly from LDs to mitochondria and maximizing the fatty acid oxidation (FIG. 4H) [56].

Importantly, the dependence of cancer cells on fatty acid oxidation is increased in nutrient-depleted conditions [58]. In support of this, oxidation of endogenous fatty acids significantly contributed to the oxidative phosphorylation rate in MK2206+CYT387 co-treated cells compared to control (>2.5-fold increase, p<0.0001) (FIG. 4I). This suggested that cellular lipid remodeling by the autophagy-lysosome system may supply a considerable fraction of the intracellular lipids-fatty acids irrespective of their external availability.

### Inhibiting PLA2 Activity Decreases Autophagy-Induced Lipid Droplets, Limits Oxidative Phosphorylation, and Increases Apoptosis

Our data implicated hydrolysis of phospholipids as a critical mechanism for the generation of lysophospholipids and fatty acids for fatty acid oxidation in treated RCC cells, and therefore inhibition of this enzymatic activity would negatively impact oxidative phosphorylation and subsequently limit the survival of these cells. To directly test this, we added the PLA2 inhibitor oleyloxyethylphosphocholine (OOEPC) [59] to CYT387, MK2206 and CYT387-MK2206 co-treated cells and measured LD numbers. Consistent with its rate-limiting role, addition of OOEPC significantly reduced the LD abundance in CYT387, MK2206 and CYT387-MK2206 co-treated cells (FIG. 5A, 5B).

To directly test the metabolic impact of OOEPC treatment, we first assessed changes in the OCR. We observed a marked decrease in the basal OCR when OOEPC was added to the CYT387-MK2206 combination. Importantly, the addition of OOPEC significantly reduced the spare respiratory capacity (the quantitative difference between maximal uncontrolled OCR and initial basal OCR), indicating that the inhibition of PLA2 decreases mitochondrial oxidation by reducing fatty acid supply, and impedes the cells' capacity to respond to increased energetic demands (FIG. 5C, 5D). Next, by plotting OCR versus ECAR, we determined the effect of PLA2 inhibition by OOEPC on CYT387-MK2206 treated tumors; this measurement highlighted that untreated ACHN human RCC cells have higher OXPHOS and glycolysis compared to CYT387-MK2206 co-treated cells (FIG. 5E). The addition of OOEPC markedly decreased ECAR and OCR in ACHN cells, indicating that these treatments diminished the overall metabolic activity of the cancer cells.

This observed reduction in bioenergetic metabolism led us to determine whether PLA2 inhibition would have an impact on proliferation and apoptosis. Co-treatment with OOEPC had minimal additional effect on proliferation (FIG. 5F). By contrast, the addition of OOEPC significantly increased apoptosis, consistent with its ability to reverse autophagy supplied fatty acids that enable survival (FIG. 5G). To further verify that PLA2 inhibition impacted cancer cell survival, we tested a distinct PLA2 inhibitor, varespladib, which has been clinically developed for cardiovascular diseases [60]. Similar to OOEPC, the addition of varespladib to CYT387-MK2206 treated cells decreased LDs and increased apoptosis (FIG. 5H, 5I, 5J).

Taken together, these data indicate that treatment-induced autophagy provides lysophospholipids and free fatty acids to maintain cancer cell survival despite nutrient depletion.

### DISCUSSION

In this study, we show that cancer cells when acutely exposed to small molecule inhibitors activate the autophagic process to ensure early and lasting metabolic adaptations designed to enhance survival in a nutrient-depleted environment. One of the first changes we observed was the metabolic switch from glycolysis to oxidative phosphorylation when glucose became limiting due to treatment. Likewise, the coordinate activation of AMPK signaling to ensure protective redox homeostasis to mitigate increased ROS produced by oxidative phosphorylation. Finally, we demonstrated activation of autophagy-mediated membrane glycerophospholipid metabolism with subsequent fatty acid oxidation to generate energy. Accordingly, we find that therapy-induced autophagy purposefully harnesses core biological processes to secure tumor cell fitness and survival.

Screening identified several structurally different Janus-family kinase inhibitors which inhibited mTORC1 and induced autophagic flux. To date, Janus kinase inhibitors have been approved for and/or are undergoing late stage clinical trials in MPN, including the focus of this study, CYT387 (Momelotinib®) [14,15]. However, complete cytogenetic or molecular responses with JAK inhibitors have not been observed, with clinical benefit mainly resulting from improved performance status due to reduced cytokine levels rather than the elimination of cancer cells [61,62]. Therefore, our finding that JAK inhibitors induce autophagy in both solid tumors and MPN cells which then maintain residual disease potentially through the hydrolysis of phospholipids may offer an explanation as to why this class of inhibitors have not been able to eradicate cancer cells and effect durable responses.

This study further addresses the wider question of how cancer cells survive despite the inhibition of mTOR, an evolutionary conserved master regulator of cell metabolism, proliferation, growth and survival, and AKT, a committed pro-survival kinase that positively regulates these same processes in both normal and cancer cells [12,13]. Undoubtedly, the combination of attenuated proliferation signals, nutrient depletion and metabolic competition for remaining nutrients kills many cells. Accordingly, our data demonstrates that glucose, which is tightly regulated by the PI3K-AKT-mTOR pathway at multiple steps became limiting with treatment, with resultant decrease in glycolysis [27, 63, 64]. However, the very same conditions that give rise to these nutrient-deprived microenvironments also induced autophagy. Consequently, the autophagic catabolism of membrane phospholipids provides a ready source of free fatty acids that maintains respiration in subpopulations of cancer cells, therefore enabling their survival in a low glucose environment. The increase in fatty acid oxidation and oxidative phosphorylation requires redox homeostasis, and this is provided by the concomitant activation of AMPK, which increases NADPH with subsequent mitigation of ROS. Collectively, treatment enforced metabolic reprogramming supports cancer cell fitness by providing fatty acids and NADPH to maximize survival.

We demonstrate that the withdrawal of growth factor signaling and nutrients induce the production of LDs, increase mitochondria number and increase the physical proximity between LDs and mitochondria. Although the increase in LDs and fatty acid oxidation is seemingly paradoxical, e.g. akin to a “futile” cycle [65-67], our findings suggest that autophagic digestion of phospholipids, with subsequent hydrolysis within the autolysosome provides LDs with a constant supply of lipids, which can then be trafficked to the mitochondria [56]. Autophagy was necessary for the development of lipid droplets, as no increase in lipid droplets occurred in mouse embryonic fibroblasts deficient for the essential autophagy gene, ATG5 when treated with CYT387, MK2206 and the CYT387-MK2206 combination.

Since the rate of autophagic release of fatty acids does not match the rate of mitochondrial consumption, these LDs serve a dual purpose: first, as a buffer to reduce lipotoxicity by storing lipid intermediates and second, to transport these lipids to the mitochondria [56,68]. Consequently, these energy-strapped residual cancer cells increase fatty acid oxidation, as it is the most energetically efficient way to generate ATP. Long-lived cell types like cardiac myocytes and memory T-cells [69, 70] depend on fatty acid metabolism for survival, and we see this as yet another example of cancer cells hijacking normal physiological processes to their benefit.

Prior work has shown that hypoxic and Ras-transformed pancreatic cancer cells support growth by scavenging unsaturated fatty acids from extracellular lysophospholipids through macropinocytosis [71]. Our data suggest a complementary model where in the setting of pharmacologically-induced nutrient depletion (in this case, glucose), the autophagic-lysosomal hydrolysis of phospholipids provides lysophospholipids and easily accessible free fatty acids that are trafficked into LDs, from where they can be transferred into mitochondria for fatty acid oxidation. Accordingly, inhibition of phospholipid hydrolysis by PLA2 inhibitors reduced LD abundance and markedly increased apoptosis in treated cells. Moreover, although we only addressed the metabolic ramifications of increased lysophospholipids and arachidonic acid, these lipids are the source of bioactive eicosanoids that have important roles in proliferation, angiogenesis and cancer progression, and so the development of clinically active PLA2 inhibitors extends its therapeutic utility [72].

### Materials and Methods

### Cell Lines

ACHN, Caki-1, RCC10, SN12C, TK-10, U031, 786-0, UKE-1, SET-2, and HEL were used in this study and were obtained from the ATCC. MEF ATG5 wild type and ATG5−/− were a kind gift from Jay Debnath (UCSF). Cell lines were maintained in Dulbecco's modified Eagle's medium (DMEM) supplemented with 10% fetal bovine serum (FBS) at 37° C. in a 5% CO2 incubator.

### Patient Tumor Ex Vivo Organotypic Culture

Tumor tissue samples were collected at the time of surgical removal for consented patients and transported in IMEM+FBS+PS. Tissue was sliced into thin sections using a surgical knife. Sections were cultured on an organotypic insert (EMD#PICMORG50) for 24 hours in IMEM+10% FBS+1% PS+50 ug/ml holo-transferrin with drug. A section of each tumor was immediately fixed in 10% buffered formalin to confirm tissue viability. After culture, treated tissue sections were fixed in 10% buffered formalin and embedded in paraffin. Paraffin embedded tumors were evaluated for morphology (H&E) and immunofluorescent signaling.

### Cell Viability and Apoptosis Analysis

Cell viability assays were performed by plating 2000 cells/well in 96-well plates in triplicate and treating the following day with the indicated agent. The experiment was continued for 3 days and then cell viability was determined using CellTiter-Glo assay kit (Promega).

The assay was measured using a luminometer. The effect of CYT387, MK2206 and the CYT387+MK2206 combination on cell number was assessed as fold of DMSO-treated control cells. Experimental results are the average of at least three independent experiments.

Apoptosis was determined using Caspase 3/7-Glo assay kit (Promega) following the manufacturer's instructions. Briefly, 2000 cells per well were plated in 96 well plates and cultured for 72 h. Cells were treated with CYT387, MK2206 and the combination of CYT387 and MK2206 for 72 h, and then 100 μl reagents were added to each well and incubated for 30 min at room temperature. Caspase 3/7 activity was measured using a luminometer. Luminescence values were normalized by cell numbers. The effect of CYT387, MK2206 and the CYT387+MK2206 combination on caspase 3/7 activation was assessed as fold of DMSO-treated control cells.

### High Content Imaging

A 7-point dilution series of 116 small molecule inhibitors covering a 1000× concentration range were plated into three, 384-well plates using the EP Motion automated dispensing system. Control wells with equal volumes of DMSO were included as negative controls. ACHN cells were grown, trypsinized counted, and plated directly into warm drug plates using Multidrop combi dispenser. Plates were incubated for 72 hr and subsequently imaged on the Olympus ScanR Platform at 10× magnification performing 4 images per well in 384 well plates. Single-cell nuclear and cytoplasmic fluorescent intensities were calculated using the Olympus ScanR Analysis Software: the DAPI-positive region of each cell was used as a boundary to quantitate nuclei counts for analysis of cell growth and integrated nuclear DNA staining intensity was used for cell cycle analysis. A 10-pixel extension of the nuclear region (and not including the nuclear region) was used to quantitate cytoplasmic signal of immunofluorescent staining of p62 protein and phosphorylation of S6. Mean signal intensity of each marker in all cells per well was used as the metric for cytoplasmic marker expression (average intensity of pS6 and p62). Unsupervised hierarchical clustering was used to identify compounds that produced similar pS6 and p62 dose response phenotypes after treatment.

### Western Blotting

Cells were plated in 6 well dishes and treated the following day with the indicated agents. Treatments were for 24 hours, after which cells were washed with ice cold PBS and lysed with RIPA buffer (Sigma). Phosphatase inhibitor cocktail set II and protease inhibitor cocktail set III (EMD Millipore) were added at the time of lysis. Lysates were centrifuged at 15,000 g×10 min at 4 degrees C. Protein concentrations were calculated based on a BCA assay (Thermo Scientific) generated standard curve. Proteins were resolved using the NuPAGE Novex Mini Gel system on 4% to 12% Bis-Tris Gels (Invitrogen). For western blotting, equal amounts of cell lysates (15-20 μg of protein) were resolved with SDSPAGE, and transferred to membranes. The membrane was probed with primary antibodies, washed, and then incubated with corresponding fluorescent secondary antibodies and washed. The fluorescent signal was captured using LI-COR (Lincoln, Nebr.) Odyssey Imaging System, and fluorescent intensity was quantified using the Odyssey software where indicated. The following antibodies were used for Western blots: p-S6 (S240/244), S6, LC3B, p-Akt(S473), p-Akt(T308), Akt, cleaved caspase3 (Cell Signaling Technologies). p-Stat3 (Y705), Stat3 and β-actin (AC15) (Abcam). Ki67 (Dako) and cleaved caspase3 (Cell Signaling Technologies) were used for immunohistochemistry. MK2206 and CYT387 for in vitro and in vivo use were purchased from LC Labs and ChemieTek, respectively. BX795 and GDC0941 were purchased from Sigma.

### In Vivo Xenograft Studies

6-week old mice were utilized for human renal cell carcinoma xenografts. For both ACHN and SN12C cell lines 2×106 cells were diluted in 50 μl of PBS and 50 μl of Matrigel (BD Biosciences) and were injected subcutaneously into the right and left flank of each mouse. Tumors were monitored until they reached an average size of 50-80 mm3 (approximately 2 weeks), at which point treatments were begun. CYT387 (50 mg/kg/day) was administered by oral gavage 5 day/week. MK2206 (60 mg/kg/day) were administered by oral gavage 2-3 day/week. CYT387 was dissolved in NMP/Captisol (Cydex) and MK2206 was dissolved in Captisol (Cydex). Tumors and mouse weights were measured twice weekly. At least 6-8 mice per treatment group were included. All mice were euthanized using CO2 inhalation followed by cervical dislocation per institutional guidelines at Oregon Health and Science University. Experiments were approved by the Institutional Animal Care and Use Committee at OHSU.

### Phosphoproteomics Screen and Data Analysis

Enriched phospho-peptides were digested with trypsin and analyzed by mass spectroscopy following the published “Cell Signaling Technology” protocol [28-30].

### Mass Spectrometry Data Analysis

MS raw files were analyzed via MaxQuant version 1.5.3.30 [73] and MS/MS fragmentation spectra were searched using Andromeda [74] against human canonical and isoform sequences in Swiss-Prot (downloaded in September 2016 from http://uniprot.org) [75]. Quantitative phosphopeptide data were log₁₀ transformed and missing data were imputed before applying quantile normalization as previously described [76]. Hierarchical clustering was performed on the Cluster 3.0 program [77], using distance that is based on the Pearson correlation and applying pairwise average linkage analysis. Java Treeview was used to visualize clustering results [78].

### Kinase Substrate Enrichment Analysis

Kinase substrate enrichment analysis (KSEA) was performed as previously described [40]. Briefly, the phosphopeptides were rank ordered by fold change, on average, between CYT387 treatment and control and the enrichment score was calculated using the Kolmogorov-Smirnov statistic. Permutation analysis was conducted to calculate statistical significance. The normalized enrichment score was calculated by dividing the enrichment score by the average of the absolute values of all enrichment scores from the permutation analysis.

### DAVID Pathway Analysis

To generate an appropriate list for use in DAVID [41,42], phosphopeptides were initially filtered with FDR <0.20. Phosphopeptides that were 1.5-fold enriched, on average, in either CYT387 treatment or no treatment were selected. Enrichment for a phosphopeptide was reversed if a functional annotation [38] indicates protein activity inhibition. To reduce complexity of this list, if multiple phosphopeptides map to a gene, then the most enriched phosphopeptide was selected. The only exception made was if a functional annotation exists for one or more of the phosphopeptides, in which case the most enriched annotated phosphopeptide would be selected. If multiple phosphopeptides mapped to the same gene and had enrichment values that fell into both CYT387 treatment and no treatment, then those phosphopeptides and the corresponding gene were removed from the list to be analyzed. We inputted into DAVID the genes in the CYT387 treatment enriched group to examine KEGG pathways more active with CYT387 treatment.

### Phospho-Receptor Tyrosine Kinase Array

The human phospho-receptor tyrosine kinase (phospho-RTK) array kit was purchased from Cell Signaling Technologies, and screened according to the manufacturer's protocol, with 150 μg of protein being used for each experiment. Signal intensity was calculated using LI-COR (Lincoln, Nebr.) Odyssey Imaging System, and fluorescent intensity was quantified using the Odyssey software where indicated.

### Metabolomic Profiling of Cancer Cells

Metabolomic data and SRM transitions were performed as previously described [79]. Briefly, 2 million cells were plated overnight, serum starved for 2 hours prior to harvesting, after which cells were washed twice with PBS, harvested by scraping, and flash frozen. For nonpolar metabolomic analyses, flash frozen cell pellets were extracted in 4 mL of 2:1:1 chloroform/methanol/PBS with internal standards dodecylglycerol (10 nmoles) and pentadecanoic acid (10 nmoles). Organic and aqueous layers were separated by centrifugation, and organic layer was extracted. Aqueous layer was acidified with 0.1% formic acid followed by re-extraction with 2 mL chloroform. The second organic layer was combined with the first extract and dried under nitrogen, after which lipids were resuspended in chloroform (120 μl). A 10 μl aliquot was then analyzed by both single-reaction monitoring (SRM)-based LC-MS/MS or untargeted LC-MS. For polar metabolomic analyses, frozen cell pellets were extracted in 180 μl of 40:40:20 acetonitrile/methanol/water with internal standard d3 N15-serine (1 nmole).

Following vortexing and bath sonication, the polar metabolite fraction (supernatant) was isolated by centrifugation. A 20 μl aliquot was then analyzed by both single-reaction monitoring (SRM)-based LC-MS/MS or untargeted LC-MS. For the SRM transitions where we monitor the transition of parent masses to the loss of the headgroup (e.g. loss of phosphocholine from phosphatidylcholine), we have ascertained the acyl chain specificities from previously described procedures [80]. For phospholipids such as PCs and PEs, we ascertained fatty acid acyl chain composition from phospholipids using a mobile phase containing both ammonium hydroxide and formic acid and monitored the fatty acid fragmentations from [M H+HCO2H] m/z at 40 V collision energy in negative ionization mode. For other phospholipids such as PAs and PIs, we monitored the fatty acid fragmentations from [MH] m/z at 40 V collision energy in negative ionization mode in mobile phase containing just ammonium hydroxide. For the lipids that we have measured in this study, the designated acyl chains represent the primary fatty acids that were on the lipid backbone. However, this method is less sensitive than monitoring the loss of headgroup from the phospholipid, and thus we used SRM transitions for many phospholipids where we monitored for loss of headgroups (e.g. PCs, PEs, PSs, PAs, PIs).

Relative levels of metabolites were quantified by integrating the area under the curve for each metabolite, normalizing to internal standard values, and then normalizing to the average values of the control groups [49].

### Reactive Oxygen Species (ROS) Detection

ROS levels were measured with Cellrox Deep Red (Molecular Probes). Cell were plated in a 96 well clear bottom with black sides cell culture plate. After adhering for 24 hours, cells were treated with CYT387 2 μM, MK2206 10 μM and CYT387 2 μM+MK2206 10 μM. The complete media+drug was removed after 24 hours and replaced with 5 μM of Cellrox Deep Red in media. Cells were incubated for 30 min at 370 C then washed with PBS. Fluorescence signal was detected using a Bioteck Cytation 5 plate reader. Data was analyzed using Prism software.

### Cellular Respiration

Oxygen consumption and extracellular acidification rates were carried out in a XF96 Seahorse Analyzer (Seahorse Bioscience, Billerica, Mass., USA). Cells were plated in the wells of 96-well plates (8×103 cells/well; XF96 plates, Seahorse Bioscience, North Billerica, Mass.) and incubated at 37° C. overnight. The next day, cells were treated with indicated drugs for 24 hours and then the medium was changed to XF Assay Medium and loaded with glucose, oligomycin, and 2-DG, respectively, as manufacture's recommendation.

### Immunohistochemistry

Immunostaining was performed following deparaffinization and rehydration of slides. Antigen retrieval was performed in a pressure cooker using citrate buffer (pH 6.0) for 4 min. Nonspecific binding was blocked using Vector mouse IgG blocking serum 30 min at room temperature. Samples were incubated at room temperature with rabbit monoclonal antibodies pS6 (CST #5364) cleaved caspase 3 (CST#9661), and Ki67 (Dako#M7240).

Slides were developed with Vector Immpress rabbit IgG (#MP7401) and Vector Immpress mouse IgG (#MP7400) for 30 min at room temperature. Chromogenic detection was performed using Vector Immpact DAB (#SK4105) for 3 min. Slides were counterstained with hematoxylin. A 3DHistech MIDI Scanner (Perkin Elmer) was used to capture whole slide digital images with a 20× objective. Images were converted to into MRXS files and computer graphic analysis was completed using inForm 1.4.0 Advanced Image Analysis Software (Perkin Elmer).

### Morphological and IF Evaluation

H&E slides of formalin fixed, paraffin embedded tissue was used to assess morphological integrity of tumor samples. Once integrity was confirmed, immunofluorescent analysis was performed for p-S6 (1:500 CST), p-AKT (1:200 CST) and LC3B (1:250 CST). Four micron sections were cut, de-paraffinized and rehydrated. Antigen retrieval was performed using citrate for 4 min in a pressure cooker. Slides were blocked using 2.5% normal goat serum for 30 min then incubated in primary antibody for 1 hr followed by secondary antibody mouse anti-rabbit alexa 488 (1:1000) for 30 min. Slides were rinsed in PBS, air dried, and coverslipped using Dako mounting media with Dapi.

### Lipid and Mitochondrial Staining

Cells were grown on coverslips then treated with drug for 24 hours. Cells were fixed in 4% paraformaldehyde for 15 min, rinsed with PBS. Cells were washed with a 1% saponin solution for 15 min at room temperature then washed several times in PBS to remove detergent. Cells were then incubated in Bodipy (ThermoFisher #D3922) at a final concentration of 1 uM for 10 min. Bodipy was removed and slides were rinsed with PBS then air dried and mounted on slides using Dako mounting media with Dapi.

To detect mitochondrial levels in treated cells, cells were grown on coverslips for 4 hours. Mitotracker Orange (ThermoFisher # M7511) was diluted in media with drug at a final concentration of 1 uM and incubated overnight. Media was removed and cells were fixed with 4% paraformaldehyde for 15 min. Cells were rinsed 2×5 min in PBS. Cells were then incubated in cold acetone at −20C for 10 min. Acetone was removed, cells were washed in PBS, air dried and mounted on slides with Dako mounting media with dapi. A 3DHistech MIDI Scanner (Perkin Elmer) was used to capture whole slide digital images with a 20× objective. Images were converted to into MRXS files and computer graphic analysis was completed using in Form 1.4.0 Advanced Image Analysis Software (Perkin Elmer).

### MDC Staining

Slides were plated on coverslips and allowed to adhere for 24 hours. After adherence, cells were treated with drug for 24 hours. After treatment, drug was removed and cells were washed once in PBS. Cells were labeled with a 50 mM concentration of autofluorescent marker monodansylcadaverine (MDC) in PBS for 10 min at 37 C. Cells were fixed in 4% formaldehyde for 15 min at room temperature. Cells were washed in PBS 2×5 min, and mounted on slides using Dako mounting media with dapi. Coverslips were sealed with clear nail polish and imaged with 3DHistech MIDI Scanner as described above.

### Statistical Analyses

Mouse tumor size was analyzed by 2-way ANOVA with time and drug as factors, using GraphPad Prism. Mouse weight during treatment was analyzed by repeated measures 2-way ANOVA, with time and drug as factors. A P value less than 0.05 was considered statistically significant. Immunohistochemistry: P-values were calculated using one-way ANOVA, with Bonferroni's multiple comparison test. * denotes P<0.05, ** denotes P<0.01, and *** denotes P<0.001 throughout this disclosure. Metabolite fold-changes were computed and visualized in Python script, using the openpyxl package (for importing Excel files) and the matplotlib package (for visualizing fold changes).

Information concerning this invention is published online on Nov. 14, 2017 under the title Metabolic reprogramming ensures cancer cell survival despite oncogenic signaling Blockade, Genes & Dev., doi:10.1101/gad.305292.117, the contents of which are incorporated herein by reference in their entirety.

### Myelodysplastic Syndrome

Also provided is a method of inhibiting dysplastic or abnormal cell survival in a human experiencing myelodysplastic syndrome treatment-induced autophagy, the method comprising administering to a human in need thereof a pharmaceutically effective amount of a phospholipase A2 inhibitor, or a pharmaceutically acceptable salt thereof. In some embodiments, the myelodysplastic syndrome treatment-induced autophagy results from the human being treated for myelodysplastic syndrome with a JAK inhibitor. In some embodiments, the JAK inhibitor resulting in the treatment-induced autophagy is selected from the group of momelotinib and ruxolitinib, or a pharmaceutically acceptable salt thereof. In some embodiments, the JAK inhibitor resulting in the treatment-induced autophagy is selected from the group of momelotinib and ruxolitinib, tofacitinib (CP-690550), azd1480, and fedratinib (SAR302503), or a pharmaceutically acceptable salt thereof.

Also provided is a method of treatment of myelodysplastic syndrome in a subject, the method comprising administering to a subject in need thereof:
a) a pharmaceutically effective amount of a JAK inhibitor, or a pharmaceutically acceptable salt thereof; and
b) a pharmaceutically effective amount of a phospholipase A2 inhibitor, or a pharmaceutically acceptable salt thereof.

Also provided is a method of treatment of myelodysplastic syndrome in a subject, the method comprising administering to a subject in need thereof:
a) a pharmaceutically effective amount of a JAK inhibitor selected from the group of momelotinib and ruxolitinib, tofacitinib (CP-690550), azd1480, and fedratinib (SAR302503), or a pharmaceutically acceptable salt thereof, or a pharmaceutically acceptable salt thereof; and
b) a pharmaceutically effective amount of a phospholipase A2 inhibitor selected from the group of anagrelide, cilostazol, varespladib, Darapladib, ulobetasol, oleyloxyethyl phosphorylcholine, cytidine 5-prime-diphosphocholine, U-73122, quinacrine, quercetin dihydrate, chlorpromazine, aristolochic acid, cynnamycin, MJ33, ETYA, N-(p-amylcinnamoyl)anthranilic acid, isotetrandrine, quinacrine dihydrochloride dihydrate, YM 26734, dihydro-D-erythro-sphingosine, PACOCF3, ONO-RRS-082, Luffariellolide, RSC-3388, LY 311727, OBAA, AX 048, 2-Hydroxy-1,1,1,-trifluoro-6,9,12,15-heneicosatetraene, 2-oxo-1,1,1-Trifluoro-6,9-12,15-heneicosatetraene, 2-oxo-6,9,12,15-Heneicosatetetraene, (E)-6-(Bromomethylene)tetrahydro-3-(1-naphthalenyl)-2H-pyran-2-one, 4,7,10,13-Nonadecatetraenyl fluorophosphonic acid methyl ester, N-[6-(4-Chlorophenyl)hexyl]-2-oxo-4-[(S)-(phenylmethyl)sulfinyl]-1-azetidineacetamide, N-[6-(4-Chlorophenyl)hexyl]-2-oxo-4-[(R)-(phenylmethyl)sulfinyl]-1-azetidineacetamide, Palmityl trifluoromethylketone, and (S)-bromoenol lactone, darapladib, N-(2-diethylamino-ethyl)-2-[2-(4-fluoro-benzylsulfanyl)-4-oxo-4,5,6,7-tetra hydro-cyclopenta pyrimidin-1-yl]-N-(4′-trifluoromethyl-biphenyl-4-ylmethyl)-acetamide, SB435495, GSK-2647544, varespladib, mepacrine bromophenylbromide, darapladib, N-(2-diethylamino-ethyl)-2-[2-(4-fluoro-benzylsulfanyl)-4-oxo-4,5,6,7-tetra hydro-cyclopenta pyrimidin-1-yl]-N-(4′-trifluoromethyl-biphenyl-4-ylmethyl)-acetamide, SB435495, GSK-2647544, varespladib, and mepacrine bromophenylbromide; or a pharmaceutically acceptable salt thereof.

Further provided is a method of treatment of myelodysplastic syndrome in a subject, the method comprising administering to a subject in need thereof:
a) a pharmaceutically effective amount of a JAK inhibitor selected from the group of momelotinib, ruxolitinib, tofacitinib (CP-690550), azd1480, and fedratinib (TGI101348, SAR302503), or a pharmaceutically acceptable salt thereof, or a pharmaceutically acceptable salt thereof; and
b) a pharmaceutically effective amount of a phospholipase A2 inhibitor selected from the group of anagrelide and cilostazol, or a pharmaceutically acceptable salt thereof.

Tofacitinib may be administered orally in the methods herein at a daily dose of from about 1 mg to about 20 mg. In some embodiments, tofacitinib may be administered at a dose of from about 5 mg to about 10 mg once or twice daily.

Fedratinib may be administered in the methods herein at a dose of from about 50 mg to about 1,000 mg daily. In some embodiments fedratinib is administered at a daily dose of from about 100 mg to about 750 mg. In separate embodiments, fedratinib is administered daily at a dose selected from about 100 mg, about 200 mg, about 300 mg, about 400 mg, and about 500 mg.

Further provided is a method of treatment of myelodysplastic syndrome in a subject, the method comprising administering to a subject in need thereof:
a) a pharmaceutically effective amount of a JAK inhibitor selected from the group of momelotinib and ruxolitinib, or a pharmaceutically acceptable salt thereof, or a pharmaceutically acceptable salt thereof; and
b) a pharmaceutically effective amount of a phospholipase A2 inhibitor selected from the group of anagrelide and cilostazol, or a pharmaceutically acceptable salt thereof.

In each of the embodiments above concerning methods of inhibiting dysplastic or abnormal cell survival in a human experiencing myelodysplastic syndrome treatment-induced autophagy or of treating myelodysplastic syndrome there are additional embodiments in which the human in need thereof is also administered additional therapeutic agents. In some embodiments the additional therapeutic agent is a hypomethylating agent, such as azacitidine or decitabine.

Azacitidine may be administered in the methods herein by injection at a daily dose of from about 10 mg/m² to about 150 mg/m². In some embodiments, azacitidine is administered at a daily dose of from about 25 mg/m² to about 125 mg/m². In some embodiments, azacitidine is administered at a daily dose of from about 50 mg/m² to about 100 mg/m². In some embodiments, azacitidine is administered at a daily dose of about 75 mg/m².

Decitabine may be administered in the methods herein by injection at a daily dose of from about 5 mg/m² to about 50 mg/m². In some embodiments, decitabine is administered at a daily dose of from about 10 mg/m² to about 40 mg/m². In some embodiments, decitabine is administered over a 72-hour period at a daily dose of from about 10 mg/m²/day to about 40 mg/m²/day.

In other embodiments the additional therapeutic agent is an immunomodulating drug, such as lenalidomide. In other embodiments, lenalidomide may be administered at a daily dose of from about 2.5 mg to about 50 mg. In separate embodiments, lenalidomide may be administered at daily doses of from about 2.5 mg to about 25 mg, from about 2.5 mg to about 20 mg, from about 2.5 mg to about 15 mg, from about 2.5 mg to about 10 mg, and from about 5 mg to about 15 mg.

In other embodiments the additional therapeutic agent is cytarabine alone or cytarabine in combination with one or both of idarubicin and daunorubicin. In those methods, cytarabine may be administered intravenously at a rate of from about 0.1 gm/m²/day to about 2.0 gm/m²/day for from about 1 day to about 4 days. In other embodiments, cytarabine may be administered intravenously at a rate of from about 0.2 gm/m²/day to about 1.5 gm/m²/day for from about 1 day to about 4 days. In some embodiments, cytarabine is administered at the daily doses listed above for from 3 to 4 days.

Daunorubicin may be administered intravenously in the methods herein at a daily dose rate of from about 30 mg/m² to about 150 mg/m². In other embodiments, daunorubicin may be administered intravenously in the methods herein at a daily dose rate of from about 50 mg/m² to about 100 mg/m².

In other embodiments the additional therapeutic agent is an immune system suppressing agent, such as cyclosporine and anti-thymocyte globulin. Cyclosporine may be administered orally in the methods herein at a dose of from about 1.0 mg/kg/day to about 10 mg/kg/day. In some embodiments, cyclosporine may be administered orally in the methods herein at a dose of from about 2.0 mg/kg/day to about 5 mg/kg/day.

In additional embodiments, the additional therapeutic agent may include hematopoietic growth factors, such as epoetin alpha, interleukin-3, erythropoietin, and colony stimulating factors.

### Definitions

The description herein sets forth exemplary methods, parameters and the like. It should be recognized, however, that such description is not intended as a limitation on the scope of the present disclosure but is instead provided as a description of exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The term “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound or agent, or a pharmaceutically acceptable salt or co-crystal thereof, is an amount sufficient to modulate the expression or activity in question, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of phospholipase A2, VEGF/VEGFR, mTOR, or PI3K activity.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of phospholipase A2 activity” refers to a decrease in phospholipase A2 activity as a direct or indirect response to the presence of a compound or agent, or a pharmaceutically acceptable salt or co-crystal thereof, relative to the activity of phospholipase A2 in the absence of such compound or a pharmaceutically acceptable salt or co-crystal thereof. The decrease in activity may be due to the direct interaction of the compound with phospholipase A2, or due to the interaction of the compound(s) described herein with one or more other factors that in turn affect phospholipase A2 activity. For example, the presence of the compound(s) may decrease phospholipase A2 activity by directly binding to the phospholipase A2, by causing (directly or indirectly) another factor to decrease phospholipase A2 activity, or by (directly or indirectly) decreasing the amount of phospholipase A2 present in the cell or organism. In some embodiments, the inhibition of phospholipase A2 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibiting or inhibition activity desired.

“Delaying” the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

Numerical values in the specification and claims of this application should be understood to include numerical values which are the same when reduced to the same number of significant figures and numerical values which differ from the stated value by less than the experimental error of conventional measurement technique of the type described in the present application to determine the value. All ranges disclosed herein are inclusive of the recited endpoint and independently combinable (for example, the range of “from 2 to 10” is inclusive of the endpoints, 2 and 10, and all the intermediate values).

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula I, and a guest (or co-former) molecule or molecules. In particular embodiments the pharmaceutically acceptable co-crystal of the compound of Formula I or of the compound of Formula II with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

“Subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

### REFERENCES

The following numbered references are cited throughout this disclosure by inclusion of the number reference(s) in square brackets. Each of the following references is hereby incorporated by reference in its entirety.
1. T. G. Bivona, R. C. Doebele, A framework for understanding and targeting residual disease in oncogene-driven solid cancers. Nat Med 22, 472-478 (2016).
2. L. A. Garraway, P. A. Janne, Circumventing cancer drug resistance in the era of personalized medicine. Cancer Discov 2, 214-226 (2012).
3. J. Y. Guo, E. White, Autophagy, Metabolism, and Cancer. Cold Spring Harb Symp Quant Biol, (2017).
4. J. M. Levy, A. Thorburn, Targeting autophagy during cancer therapy to improve clinical outcomes. Pharmacol Ther 131, 130-141 (2011).
5. L. Galluzzi et al., Autophagy in malignant transformation and cancer progression. EMBO J 34, 856-880 (2015).
6. J. T. Leonard et al., Targeting BCL-2 and ABL/LYN in Philadelphia chromosomepositive acute lymphoblastic leukemia. Sci Transl Med 8, 354ra114 (2016).
7. J. E. Maxson et al., Identification and Characterization of Tyrosine Kinase Nonreceptor 2 Mutations in Leukemia through Integration of Kinase Inhibitor Screening and Genomic Analysis. Cancer Res 76, 127-138 (2016).
8. J. E. Maxson et al., Oncogenic CSF3R mutations in chronic neutrophilic leukemia and atypical CML. N Engl J Med 368, 1781-1790 (2013).
9. R. J. Motzer et al., Kidney cancer, version 3.2015. J Natl Compr Canc Netw 13, 151-159 (2015).
10. V. Rogov, V. Dotsch, T. Johansen, V. Kirkin, Interactions between autophagy receptors and ubiquitin-like proteins form the molecular basis for selective autophagy. Mol Cell 53, 167-178 (2014).
11. J. Joachim, M. Jiang, N. C. McKnight, M. Howell, S. A. Tooze, High-throughput screening approaches to identify regulators of mammalian autophagy. Methods 75, 96-104 (2015).
12. M. Laplante, D. M. Sabatini, mTOR signaling in growth control and disease. Cell 149, 274-293 (2012).
13. P. Bose, S. Verstovsek, Drug Development Pipeline for Myeloproliferative Neoplasms: Potential Future Impact on Guidelines and Management. J Natl Compr Canc Netw 14, 1613-1624 (2016).
14. A. B. Patel, N. A. Vellore, M. W. Deininger, New Strategies in Myeloproliferative Neoplasms: The Evolving Genetic and Therapeutic Landscape. Clin Cancer Res 22, 1037-1047 (2016).
15. E. F. Winton, V. Kota, Momelotinib in myelofibrosis: JAK1/2 inhibitor with a role in treating and understanding the anemia. Future Oncol 13, 395-407 (2017).
16. B. G. Bagca et al., Ruxolitinib induces autophagy in chronic myeloid leukemia cells. Tumour Biol 37, 1573-1579 (2016).
17. S. C. Meyer, R. L. Levine, Molecular pathways: molecular basis for sensitivity and resistance to JAK kinase inhibitors. Clin Cancer Res 20, 2051-2059 (2014).
18. S. Shen et al., Cytoplasmic STAT3 represses autophagy by inhibiting PKR activity. Mol Cell 48, 667-680 (2012).
19. J. Debnath, Detachment-induced autophagy during anoikis and lumen formation in epithelial acini. Autophagy 4, 351-353 (2008).
20. S. Turcotte et al., A molecule targeting VHL-deficient renal cell carcinoma that induces autophagy. Cancer Cell 14, 90-102 (2008).
21. F. Cecconi, B. Levine, The role of autophagy in mammalian development: cell makeover rather than cell death. Dev Cell 15, 344-357 (2008).
22. C. Fung, R. Lock, S. Gao, E. Salas, J. Debnath, Induction of autophagy during extracellular matrix detachment promotes cell survival. Mol Biol Cell 19, 797-806 (2008).
23. D. J. Klionsky et al., Guidelines for the use and interpretation of assays for monitoring autophagy (3rd edition). Autophagy 12, 1-222 (2016).
24. R. M. Perera et al., Transcriptional control of autophagy-lysosome function drives pancreatic cancer metabolism. Nature 524, 361-365 (2015).
25. I. Garcia-Cao et al., Systemic elevation of PTEN induces a tumor-suppressive metabolic state. Cell 149, 49-62 (2012).
26. K. Duvel et al., Activation of a metabolic gene regulatory network downstream of mTOR complex 1. Mol Cell 39, 171-183 (2010).
27. J. L. Yecies, B. D. Manning, Transcriptional control of cellular metabolism by mTOR signaling. Cancer Res 71, 2815-2820 (2011).
28. A. Moritz et al., Akt-RSK-S6 kinase signaling networks activated by oncogenic receptor tyrosine kinases. Sci Signal 3, ra64 (2010).
29. J. Rush et al., Immunoaffinity profiling of tyrosine phosphorylation in cancer cells. Nat Biotechnol 23, 94-101 (2005).
30. G. Zhuang et al., Phosphoproteomic analysis implicates the mTORC2-FoxO1 axis in VEGF signaling and feedback activation of receptor tyrosine kinases. Sci Signal 6, ra25 (2013).
31. B. D. Manning, A. R. Tee, M. N. Logsdon, J. Blenis, L. C. Cantley, Identification of the tuberous sclerosis complex-2 tumor suppressor gene product tuberin as a target of the phosphoinositide 3-kinase/akt pathway. Mol Cell 10, 151-162 (2002).
32. P. P. Roux, B. A. Ballif, R. Anjum, S. P. Gygi, J. Blenis, Tumor-promoting phorbol esters and activated Ras inactivate the tuberous sclerosis tumor suppressor complex via p90 ribosomal S6 kinase. Proc Natl Acad Sci USA 101, 13489-13494 (2004).
33. J. Huang, B. D. Manning, A complex interplay between Akt, TSC2 and the two mTOR complexes. Biochem Soc Trans 37, 217-222 (2009).
34. L. C. Kim, R. S. Cook, J. Chen, mTORC1 and mTORC2 in cancer and the tumor microenvironment. Oncogene, (2016).
35. L. A. Julien, A. Carriere, J. Moreau, P. P. Roux, mTORC1-activated S6K1 phosphorylates Rictor on threonine 1135 and regulates mTORC2 signaling. Mol Cell Biol 30, 908-921 (2010).
36. C. C. Dibble, J. M. Asara, B. D. Manning, Characterization of Rictor phosphorylation sites reveals direct regulation of mTOR complex 2 by S6K1. Mol Cell Biol 29, 5657-5670 (2009).
37. J. A. Hutchinson, N. P. Shanware, H. Chang, R. S. Tibbetts, Regulation of ribosomal protein S6 phosphorylation by casein kinase 1 and protein phosphatase 1. J Biol Chem 286, 8688-8696 (2011).
38. P. V. Hornbeck et al., PhosphoSitePlus, 2014: mutations, PTMs and recalibrations. Nucleic Acids Res 43, D512-520 (2015).
39. Y. Yin et al., mTORC2 promotes type I insulin-like growth factor receptor and insulin receptor activation through the tyrosine kinase activity of mTOR. Cell Res 26, 46-65 (2016).
40. J. M. Drake et al., Oncogene-specific activation of tyrosine kinase networks during prostate cancer progression. Proc Natl Acad Sci USA 109, 1643-1648 (2012).
41. W. Huang da, B. T. Sherman, R. A. Lempicki, Systematic and integrative analysis of large gene lists using DAVID bioinformatics resources. Nat Protoc 4, 44-57 (2009).
42. W. Huang da, B. T. Sherman, R. A. Lempicki, Bioinformatics enrichment tools: paths toward the comprehensive functional analysis of large gene lists. Nucleic Acids Res 37, 1-13 (2009).
43. B. D. Manning, L. C. Cantley, AKT/PKB signaling: navigating downstream. Cell 129, 1261-1274 (2007).
44. D. Sarker et al., First-in-human phase I study of pictilisib (GDC-0941), a potent pan-class I phosphatidylinositol-3-kinase (PI3K) inhibitor, in patients with advanced solid tumors. Clin Cancer Res 21, 77-86 (2015).
45. C. Dangelmaier et al., PDK1 selectively phosphorylates Thr(308) on Akt and contributes to human platelet functional responses. Thromb Haemost 111, 508-517 (2014).
46. T. A. Yap et al., First-in-man clinical trial of the oral pan-AKT inhibitor MK-2206 in patients with advanced solid tumors. J Clin Oncol 29, 4688-4695 (2011).
47. S. M. Jeon, N. S. Chandel, N. Hay, AMPK regulates NADPH homeostasis to promote tumour cell survival during energy stress. Nature 485, 661-665 (2012).
48. H. R. Christofk, M. G. Vander Heiden, N. Wu, J. M. Asara, L. C. Cantley, Pyruvate kinase M2 is a phosphotyrosine-binding protein. Nature 452, 181-186 (2008).
49. S. M. Louie et al., GSTP1 Is a Driver of Triple-Negative Breast Cancer Cell Metabolism and Pathogenicity. Cell Chem Biol 23, 567-578 (2016).
50. N. Mizushima et al., Dissection of autophagosome formation using Apg5-deficient mouse embryonic stem cells. J Cell Biol 152, 657-668 (2001).
51. S. Eaton, Control of mitochondrial beta-oxidation flux. Prog Lipid Res 41, 197-239 (2002).
52. P. F. Finn, J. F. Dice, Proteolytic and lipolytic responses to starvation. Nutrition 22, 830-844 (2006).
53. J. Kerner, C. Hoppel, Fatty acid import into mitochondria. Biochim Biophys Acta 1486, 1-17 (2000).
54. M. Murakami et al., Recent progress in phospholipase A(2) research: from cells to animals to humans. Prog Lipid Res 50, 152-192 (2011).
55. A. R. Thiam, R. V. Farese, Jr., T. C. Walther, The biophysics and cell biology of lipid droplets. Nat Rev Mol Cell Biol 14, 775-786 (2013).
56. A. S. Rambold, S. Cohen, J. Lippincott-Schwartz, Fatty acid trafficking in starved cells: regulation by lipid droplet lipolysis, autophagy, and mitochondrial fusion dynamics. Dev Cell 32, 678-692 (2015).
57. C. Sztalryd, A. R. Kimmel, Perilipins: lipid droplet coat proteins adapted for tissue specific energy storage and utilization, and lipid cytoprotection. Biochimie 96, 96-101 (2014).
58. A. G. Cabodevilla et al., Cell survival during complete nutrient deprivation depends on lipid droplet-fueled beta-oxidation of fatty acids. J Biol Chem 288, 27777-27788 (2013).
59. D. A. Slatter et al., Mapping the Human Platelet Lipidome Reveals Cytosolic Phospholipase A2 as a Regulator of Mitochondrial Bioenergetics during Activation. Cell Metab 23, 930-944 (2016).
60. R. S. Rosenson et al., Effects of varespladib methyl on biomarkers and major cardiovascular events in acute coronary syndrome patients. J Am Coil Cardiol 56, 1079-1088 (2010).
61. A. M. Vannucchi et al., Ruxolitinib versus standard therapy for the treatment of polycythemia vera. N Engl J Med 372, 426-435 (2015).
62. S. Verstovsek et al., A double-blind, placebo-controlled trial of ruxolitinib for myelofibrosis. N Engl J Med 366, 799-807 (2012).
63. J. A. Engelman, J. Luo, L. C. Cantley, The evolution of phosphatidylinositol 3-kinases as regulators of growth and metabolism. Nat Rev Genet 7, 606-619 (2006).
64. H. Hu et al., Phosphoinositide 3-Kinase Regulates Glycolysis through Mobilization of Aldolase from the Actin Cytoskeleton. Cell 164, 433-446 (2016).
65. A. G. Dulloo, M. Gubler, J. P. Montani, J. Seydoux, G. Solinas, Substrate cycling between de novo lipogenesis and lipid oxidation: a thermogenic mechanism against skeletal muscle lipotoxicity and glucolipotoxicity. Int J Obes Relat Metab Disord 28 Suppl 4, S29-37 (2004).
66. H. P. Guan et al., A futile metabolic cycle activated in adipocytes by antidiabetic agents. Nat Med 8, 1122-1128 (2002).
67. X. X. Yu, D. A. Lewin, W. Forrest, S. H. Adams, Cold elicits the simultaneous induction of fatty acid synthesis and beta-oxidation in murine brown adipose tissue: prediction from differential gene expression and confirmation in vivo. FASEB J 16, 155-168 (2002).
68. R. H. Unger, G. O. Clark, P. E. Scherer, L. Orci, Lipid homeostasis, lipotoxicity and the metabolic syndrome. Biochim Biophys Acta 1801, 209-214 (2010).
69. S. Chung, D. K. Arrell, R. S. Faustino, A. Terzic, P. P. Dzeja, Glycolytic network restructuring integral to the energetics of embryonic stem cell cardiac differentiation. J Mol Cell Cardiol 48, 725-734 (2010).
70. E. L. Pearce et al., Enhancing CD8 T-cell memory by modulating fatty acid metabolism. Nature 460, 103-107 (2009).
71. J. J. Kamphorst et al., Hypoxic and Ras-transformed cells support growth by scavenging unsaturated fatty acids from lysophospholipids. Proc Natl Acad Sci US A 110, 8882-8887 (2013).
72. D. Wang, R. N. Dubois, Eicosanoids and cancer. Nat Rev Cancer 10, 181-193 (2010).
73. J. Cox, M. Mann, MaxQuant enables high peptide identification rates, individualized p.p.b.-range mass accuracies and proteome-wide protein quantification. Nat Biotechnol 26, 1367-1372 (2008).
74. J. Cox et al., Andromeda: a peptide search engine integrated into the MaxQuant environment. J Proteome Res 10, 1794-1805 (2011).
75. R. Apweiler et al., UniProt: the Universal Protein knowledgebase. Nucleic Acids Res 32, D115-119 (2004).
76. J. M. Drake et al., Phosphoproteome Integration Reveals Patient-Specific Networks in Prostate Cancer. Cell 166, 1041-1054 (2016).
77. M. B. Eisen, P. T. Spellman, P. O. Brown, D. Botstein, Cluster analysis and display of genome-wide expression patterns. Proc Natl Acad Sci USA 95, 14863-14868 (1998).
78. A. J. Saldanha, Java Treeview—extensible visualization of microarray data. Bioinformatics 20, 3246-3248 (2004).
79. R. Camarda et al., Inhibition of fatty acid oxidation as a therapy for MYCoverexpressing triple-negative breast cancer. Nat Med 22, 427-432 (2016).
80. J. Z. Long et al., Metabolomics annotates ABHD3 as a physiologic regulator of medium-chain phospholipids. Nat Chem Biol 7, 763-765 (2011).

What is claimed:

1. A method of inhibiting cancer cell survival in a human experiencing treatment-induced autophagy, the method comprising administering to a human in need thereof a pharmaceutically effective amount of a phospholipase A2 inhibitor, or a pharmaceutically acceptable salt thereof.

2. The method of claim 1, wherein the experiencing treatment-induced autophagy is resulting from the human receiving a pharmaceutical agent selected from the group of a Janus Kinase (JAK) inhibitor, VEGF/VEGFR receptor tyrosine kinase inhibitor, a protein kinase A (PKA) inhibitor, a multi-kinase inhibitor, a phosphoinositide 3-kinase (PI3K) inhibitor, a mechanistic target of rapamycin (mTOR) inhibitor, a protein kinase C (PKC) inhibitor, a mitogen-activated protein kinase kinase (MEK) inhibitor, a CDK9 inhibitor, and a proteasome inhibitor; or a pharmaceutically acceptable salt thereof.

3. The method of claim 2, wherein the phosholipase A2 inhibitor is selected from the group of anagrelide, cilostazol, varespladib, Darapladib, ulobetasol, oleyloxyethyl phosphorylcholine, cytidine 5-prime-diphosphocholine, U-73122, quinacrine, quercetin dihydrate, chlorpromazine, aristolochic acid, cynnamycin, MJ33, ETYA, N-(p-amylcinnamoyl)anthranilic acid, isotetrandrine, quinacrine dihydrochloride dihyrate, YM 26734, dihydro-D-erythro-sphingosine, PACOCF3, ONO-RRS-082, Luffariellolide, RSC-3388, LY 311727, OBAA, AX 048, 2-Hydroxy-1,1,1,-trifluoro-6,9,12,15-heneicosatetraene, 2-oxo-1,1,1-Trifluoro-6,9-12,15-heneicosatetraene, 2-oxo-6,9,12,15-Heneicosatetetraene, (E)-6-(Bromomethylene)tetrahydro-3-(1-naphthalenyl)-2H-pyran-2-one, 4,7,10,13-Nonadecatetraenyl fluorophosphonic acid methyl ester, N-[6-(4-Chlorophenyl)hexyl]-2-oxo-4-[(S)-(phenylmethyl)sulfinyl]-1-azetidineacetamide, N-[6-(4-Chlorophenyl)hexyl]-2-oxo-4-[(R)-(phenylmethyl)sulfinyl]-1-azetidineacetamide, Palmityl trifluoromethylketone, and (S)-bromoenol lactone, darapladib, N-(2-diethylamino-ethyl)-2-[2-(4-fluoro-benzylsulfanyl)-4-oxo-4,5,6,7-tetra hydro-cyclopenta pyrimidin-1-yl]-N-(4′-trifluoromethyl-biphenyl-4-ylmethyl)-acetamide, SB435495, GSK-2647544, varespladib, mepacrine bromophenylbromide, darapladib, N-(2-diethylamino-ethyl)-2-[2-(4-fluoro-benzylsulfanyl)-4-oxo-4,5,6,7-tetra hydro-cyclopentapyrimidin-1-yl]-N-(4′-trifluoromethyl-biphenyl-4-ylmethyl)-acetamide, SB435495, GSK-2647544, varespladib, and mepacrine bromophenylbromide; or a pharmaceutically acceptable salt thereof.

4. The method of claim 2, wherein the phosholipase A2 inhibitor is selected from the group of anagrelide and cilostazol, or a pharmaceutically acceptable salt thereof.

5. The method of claim 2 wherein the treatment-induced autophagy is resulting from the human receiving a phosphoinositide 3-kinase (PI3K) inhibitor, or a pharmaceutically acceptable salt thereof.

6. The method of claim 5, the method comprising administering to a human in need thereof a pharmaceutically effective amount of a phospholipase A2 inhibitor, or a pharmaceutically acceptable salt thereof, and a pharmaceutically effective amount of a PI3K inhibitor, or a pharmaceutically acceptable salt thereof.

7. The method of claim 5 wherein the PI3K inhibitors is one or more agents selected from the group of buparlisib, pictilisib, pilaralisib, coplanlisib, afuresertib, alpelisib, apitolisib, dactolisib, duvelisib, idelalisib, ipatasertib, omipalisib, perifosine, pictilisib, sapanisertib, taselisib, and umbralisib, or a pharmaceutically acceptable salt thereof.

8. The method of claim 2 wherein the treatment-induced autophagy is resulting from the human receiving an mTOR inhibitor, or a pharmaceutically acceptable salt thereof.

9. The method of claim 8, the method comprising administering to a human in need thereof a pharmaceutically effective amount of a phospholipase A2 inhibitor, or a pharmaceutically acceptable salt thereof, and a pharmaceutically effective amount of an mTOR inhibitor, or a pharmaceutically acceptable salt thereof.

10. The method of claim 2 wherein the treatment-induced autophagy is resulting from the human receiving a VEGF/VEGFR receptor tyrosine kinase inhibitor, or a pharmaceutically acceptable salt thereof.

11. The method of claim 10, the method comprising administering to a human in need thereof a pharmaceutically effective amount of a phospholipase A2 inhibitor, or a pharmaceutically acceptable salt thereof, and a pharmaceutically effective amount of a VEGF/VEGFR receptor tyrosine kinase inhibitor, or a pharmaceutically acceptable salt thereof.

12. The method of claim 11, wherein the VEGF/VEGFR receptor tyrosine kinase inhibitor is one or more agents selected from the group of pazopanib, bevacizumab, sunitinib, sorafenib, axitinib, regorafenib, ponatinib, cabozantinib, vandetanib, ramucirumab, lenvatinib, and ziv-aflibercept, or a pharmaceutically acceptable salt thereof.

13. The method of claim 2 wherein the treatment-induced autophagy is resulting from the human receiving a JAK inhibitor, or a pharmaceutically acceptable salt thereof.

14. The method of claim 13, the method comprising administering to a human in need thereof a pharmaceutically effective amount of a phospholipase A2 inhibitor, or a pharmaceutically acceptable salt thereof, and a pharmaceutically effective amount of a JAK inhibitor, or a pharmaceutically acceptable salt thereof.

15. The method of claim 14, wherein the JAK inhibitor is one or more agents selected from the group of momelotinib, ruxolitinib, tofacitinib (CP-690550), azd1480, and fedratinib (SAR302503), or a pharmaceutically acceptable salt thereof.

16. The method of claim 14, wherein the JAK inhibitor is momelotinib, or a pharmaceutically acceptable salt thereof.

17. A method of treating renal cell carcinoma in a human, the method comprising administering to the human in need thereof:
a) a pharmaceutically effective amount of a VEGF/VEGFR inhibitor, or a pharmaceutically acceptable salt thereof; and
b) a pharmaceutically effective amount of a phospholipase A2 inhibitor, or a pharmaceutically acceptable salt thereof.

18. The method of claim 17, wherein the VEGF/VEGFR inhibitor is one or more agents selected from the group of pazopanib, bevacizumab, sunitinib, sorafenib, axitinib, regorafenib, ponatinib, cabozantinib, vandetanib, ramucirumab, lenvatinib, and ziv-aflibercept, or a pharmaceutically acceptable salt thereof.

19. The method of claim 17, wherein the renal cell carcinoma is metastatic renal cell carcinoma.
